SEQUENCE LISTING (12 sequences)

SEQ ID NO: 1 - DNA, 1726 bp, Zea mays
agcccaccaccagacccgcgccaccttcgcgctccacggcagcggcatgtccgtcgcctcctcccaccgcctgcgaccgtcgccgtcgggcccggcgcggcgcccgggatccgcggcgccgcgcgtagcgttctggccagccggtgcctcgcccccgtatcgcggccgctgcaccgtcgtggcggcggcggctgccgctggcgccggcggcgaggcatcttcggcccccgtggctgtgtcggctgtcgcccccgcgggcgctgccagggacaaggtgcggcgccacacgatttcggtgttcgtcggggacgagagcggcatgatcaaccggatcgcaggggtgttcgccaggagaggctacaacatcgagtcgctcgccgtggggctcaacaaggacaaggccctcttcaccattgtcgtctccgggaccgacagggtgctcaaccaagtcatcgagcagctcaataagcttgtcaacgtcctgagtgtggaagatctatctaaagaacctcaggttgaaagagagctgatgcttataaaactaaatgttgaacctgatcagcgtcctgaggtcatggttttagttgatattttcagagcaaaagttgttgatatatctgagaaaacacttaccatggaggtagctggagatcctggcaaaattgctgcagtgcagaggaatctatggaaatttggaatcaaagaaatttgcaggacaggaaaaattgctttgagacgtgaaaggattggtgcaacagcccgtttctggcgattttctgctgcttcttacccagacctgatagaggcgttgccgaaaaatccacttacatctgtaaataggacagttaatggcagttttggtcaaccatccaatgctgggggtgatgtttatcctgtggaatcttacgagagcttatcagtgaaccatgtacttgatgctcattggggtgttctggatgatgatgatgcgactggacttcgctcgcatactctctccatccttgtgaatgactgtcctggtgtcctcaacattgtaacaggagtctttgctcgcaggggctacaatatacagagccttgctgttggcccagctgagaaggaaggcatttcgcggattacaacagttgttcctggtactgttgaatccattgagaagttagttcagcagctttacaagcttattgatgtgcacgaagttcatgacattacccactcaccttttgctgaaagggaacttatgcttattaaggtttctgtcaacactgctgctcggaaggaaatcctagatattgctgaaatcttccgagcaaaacctgttgatgtttctgaccacacagtaacgcttcagcttactggagatcttgacaagatggttgcactacaaaggttattggagccatatggcatctgtgaggtcgccagaactggacgagtagcactggtccgcgaatcgaaggtggactccaagtacctccgcggctactctcttccactgtagccttgccagtcgtgatgatgatggcaccatgaagagctcggctggttgtttatagagcagaccatctcgcgctgggttgtgttgtgcgattacaggacttgtttctttcatgtcgtgaactcccccggcctgcgtgtccaataacgcgtgttgaggctgcatgtgtattccaacgactgtcagaataagatgcatatcgtatctgtttaaaaaaaaaaaaaaaaaaaa

SEQ ID NO: 2 - protein, 483 aa, Zea mays
MSVASSHRLRPSPSGPARRPGSAAPRVAFWPAGASPPYRGRCTVVAAAAAAGAGGEASSAPVAVSAVAPAGAARDKVRRHTISVFVGDESGMINRIAGVFARRGYNIESLAVGLNKDKALFTIVVSGTDRVLNQVIEQLNKLVNVLSVEDLSKEPQVERELMLIKLNVEPDQRPEVMVLVDIFRAKVVDISEKTLTMEVAGDPGKIAAVQRNLWKFGIKEICRTGKIALRRERIGATARFWRFSAASYPDLIEALPKNPLTSVNRTVNGSFGQPSNAGGDVYPVESYESLSVNHVLDAHWGVLDDDDATGLRSHTLSILVNDCPGVLNIVTGVFARRGYNIQSLAVGPAEKEGISRITTVVPGTVESIEKLVQQLYKLIDVHEVHDITHSPFAERELMLIKVSVNTAARKEILDIAEIFRAKPVDVSDHTVTLQLTGDLDKMVALQRLLEPYGICEVARTGRVALVRESKVDSKYLRGYSLPL

SEQ ID NO: 3 - DNA, 1861 bp, Oryza sativa
ccgcataaaccctactagtgctaccatggcttaacccaaaaactaaaccgagtgccccgtgcccactgtcacacaatacagaccagccccccgtcaccttcgagctcgagccaagccaaacgatgtccgtcgcctccccgcaccatctccgcccctcgccgctggcgccggcatgccgtgctggcggcgtccccgcgcgcgccgcggcggcgcaccggccatggtgcccgcgcgtccgccgggccgtcgccgcggcctcctccggcggcggcggcggcgaggcggtgacggcggtgagcgcggcggcggtgggggcgcccgcgagcgccgcgagggacacggtgcggcgccacacgatctctgtgttcgtcggcgacgagagcgggatgataaaccggatcgccggggtgttcgccaggagagggtacaacatcgagtcgctcgccgtggggctcaacaaggacaaggccatgttcaccattgtcgtctccggcacggacagggtgctcaaccaagtcatcgagcagctcaacaagcttgtcaacgtcttgaatgtggaagatctatctaaggagccacaggttgaaagagagctgatgcttataaaaattaatgttgaaccagatcagcgtcctgaggtcatggttttagttgatattttccgagcgaaagttgttgatatttcggagaacacccttaccatcgaggtaactggagatcctggcaaaattgttgctgtgcaaaggaacctcagcaaatttgggataaaagaaatttgtagaacgggaaaaattgctttgagacgtgaaaaaattggagcaactgcccgcttctggggattttctgctgcttcttacccagatctcatagaggcattgcccaaaaattctcttcttacttctgtaaataagacagtcaatggaagttttgatcaaccatccaatgctgggggcgatgtctatcctgtggaaccttatgagggttcatccatgaaccaagtacttgatgctcactggggcgtccttgatgatgaagattcaagtggacttcgatcacatactctatccatccttgtcaatgattgccctggtgttctcaacattgttacaggggtctttgctcgcagaggctacaatatacagagtcttgctgtaggcccagctgaaaagtcaggcctttcgcgtattacaacagttgctcctggaacagatgaatccattgagaagttagttcagcagcttaacaaacttgttgatgtgcatgaggttcaagatataactcacttgccttttgctgaaagagaacttatgcttatcaaggtttctgtgaacactgctgctcggagagacatactagatattgctgaaatcttccgggcaaaatctgttgatgtttctgatcacactgttacgttacagcttactggggatctcgacaagatggttgcattacaaaggctgttggagccttatggcatctgtgaggtcgccagaacagggcgagtggcgctggtccgcgaatccggtgtcgattccaagtaccttcgtggctactcctttccgttgtaatcccaggtcttgtgagaagaaaggacagtaataaaatgcttggtcggttggttgctacctgttacagcagagtgttgtagtagagtgttgtagtcagattccgttcgttcagttatgttgtttgttatgatgctgttcttttgttgttgtttaccttcctctctgtaatgtgccaatccgctggcttcttgtccagtaaagatcatgatgcaagagttgagcctatgttttctactgacaggcaaaccaaacgtgcactcagccacttaccatgttctggaataaaaattgaa

SEQ ID NO: 4 - protein, 481 aa, Oryza sativa
MSVASPHHLRPSPLAPACRAGGVPARAAAAHRPWCPRVRRAVAAASSGGGGGEAVTAVSAAAVGAPASAARDTVRRHTISVFVGDESGMINRIAGVFARRGYNIESLAVGLNKDKAMFTIVVSGTDRVLNQVIEQLNKLVNVLNVEDLSKEPQVERELMLIKINVEPDQRPEVMVLVDIFRAKVVDISENTLTIEVTGDPGKIVAVQRNLSKFGIKEICRTGKIALRREKIGATARFWGFSAASYPDLIEALPKNSLLTSVNKTVNGSFDQPSNAGGDVYPVEPYEGSSMNQVLDAHWGVLDDEDSSGLRSHTLSILVNDCPGVLNIVTGVFARRGYNIQSLAVGPAEKSGLSRITTVAPGTDESIEKLVQQLNKLVDVHEVQDITHLPFAERELMLIKVSVNTAARRDILDIAEIFRAKSVDVSDHTVTLQLTGDLDKMVALQRLLEPYGICEVARTGRVALVRESGVDSKYLRGYSFPL

SEQ ID NO: 5 - protein, 471 aa, Triticum aestivum
MSVATAHHLRPSPPAARDRLPGCAARASSFRPLRRRGLAAGATAGGEATAAVSAVNTSAKRDPVRRHTISVFVGDESGMINRIAGVFARRGYNIESLAVGLNKDKALFTIVVSGTDRVLKQVIEQLNKLVNVLNVEDLSKEPQVERELMLIKLNVEPDQRADVMFVANVFRAKVVDISENSLTLEVTGDPGKIVAAQRNLRKFGIEEICRTGKIALRREKIGAAARFWGFSAASYPDLVEASRKNPLTSVNKTVNGSFDQPSSAGGDVYPVEPYESLSMNQVPDAHWGVLDDEDSNGLRSHTLSILVNDCPGVLNIITGVFARRGYSIQSLAVGPAEKEGISRITTVVPGTDESIEKLVQQLYKLIDVHKVDDLTDLPFAERELMVIKVSGNTAARREILDIGNIFRAECVDLSDHTVTLQLTGDLDKMVALQRLLEPYGICEVARTGRAALIRESRVGLQSTSAGYSLPL

SEQ ID NO: 6 - DNA, 31 bp, Artificial Sequence
  Description of Artificial Sequence Synthetic primer
ttcacaaggatggagagaagtatgcgagcga

SEQ ID NO: 7 - DNA, 27 bp, Artificial Sequence
  Description of Artificial Sequence Synthetic primer
acatcacccccagcattggatggttga

SEQ ID NO: 8 - DNA, 27 bp, Artificial Sequence
  Description of Artificial Sequence Synthetic primer
aagcagcagaaaatcgccagaaacggg

SEQ ID NO: 9 - DNA, 25 bp, Artificial Sequence
  Description of Artificial Sequence Synthetic primer
aacgcctctatcaggtctgggtaag

SEQ ID NO: 10 - DNA, 1502 bp, Oryza sativa
ggtaccaccgttgcaggcaatcaatacctttccggcccagttaggcagatataatgggcctcgtacgtcgcgatattgtttcgacccaaatgcaaggggctacaatgggcctcaagtatgtcgcgataacgtttcgttccaaatacaatgggctgaatgtccgttgcaatcgctcgtgctcacccggggtcttttcccctctttcctccggaacgactcgattggttccgatcagatataagggtatacatgtgtgcgtgtttaatactactacatgtttcccgtgcattaagatataggtctccaatctaaattctaacgtgtgttagaaacacgcgtgtgggtgcgtgtgttgtgtgtgatgtgagcgtggtgtgtgtacgtatacgtcctaatttgcaacttaaaaaaagatccggtgatatctctattttctgtttttttagagtcgcgtggagggagctcttggcatatggtctcgggccatctctcggtttcatctaaaaaatggcgtgctacgatttacgagaggaatcgatgacaacagtactcacatgctccacgatctcatgcttaatttttttcctcacaaaatagtcgctaaacacacgagaaacgtgacagctcccacgcacgtaaacaaaaacaaaatcgtcccaaccacttggatggagtaaagcaaacgccgcctcattgcaccacgtaacgaatcgattgacgtgcgcgcccaagaaaaacaaaactacaaaaagcaactccgaaacacgtggcatcatcttttggccgtaccgctggcacctggcatcatcacatgtatatatcgacccttcgcacatgcgtgatactacactttcacctacaaacccgagcggctcggtgacacgtacacggtacaccctccaaacccttaagaaaagaaaacctcgcaaattcagccatttcgacgcatcatcatgagttggatgggttgggtctttttatttttcgaagataaccatgttgacccatagagagcgtagacacggagccgaattgccgaaagcctccaaagcaaaagcaggcagctgtgggagccgcagccgccggggccgtcgacgtcagaccaagataccgaatatcggtcggtcccccacgccgcccagccgctgccgctgcccggccaagtagtgccccaacgcgaacgcagggccacccgtgacccatcgcgagcggatcgcgccgcggccgcgcgcggcgggtggtgtcacgctcgcactcgcactcgcacacgccgcacacgccgtctccccccaaagccaagcggcgcgcggccgcgcgggggccagcccagtaattttccaccagccgccttcgcccctccactccgcataaaccctactagtgctaccatggcttaacccaaaaactaaaccgagtgccccgtgcccactgtcacacaatacagaccagccccccgtcaccttcgagctcgagccaagccaaacgatgtccgtcgctctaga

SEQ ID NO: 11 - DNA, 1360 bp, Oryza sativa
gaattccggtgtcgattccaagtaccttcgtggctactccttttcgttgtaatcccaggtcttgtgagaagaaaggacagtaataaaatgcttggtcggttggttgctacctgttacagcagagtgttgtagtcagattccgttcgttcagttatgttgtttgttatgatgctgttcttttgttgttgtttaccttcctctctgtaatgtgccaatccgctggcttcttgtccaataaagatcatgatgcaagagttgagcctatgttttctactgacaggcaaaccaaacgtgcactcagccactcaccatgttctggaataaaaattgaatccagcgctttgcaccgtgtaaaatgttcgtaatataccatagaaaaatgtttaaaaaatcatattaatttattttacaggtttttttaaactaatactccacctaattaatcatgctctaatgggccgatagccttgtgtactactgggcctgcaggtccaacaaggctgtcggcccagtggcccacggcgatgagatgggaagcggggaatagggagaagggaagaagaatccggattctgttgcggtggttgttggaagtttggagctctcgggtcgggatcgatcgattgatcggagagggaggggaagaactccggagggggaggaggaggaggagcaccgatgacgcgggggaagcagaagatcgatgcgcagcggcggaacgcggagaagaaccagaagtcgaaggggtcccagctcgaggcccgcgccgtcggcctcaaggtcatctgccccatctgcaaggtgacgaatgaatccgtctcctcttcccttcctgcttgctaatccaaccagtagcttgtagcctgccgtattcggtattccctccccagccctaaggctagaccgctagattggatggattcatggaggattcgaggggatggctggatctttagcttcttgttatcgtctcatccacgatccacccagccctatgctcctatcccatgcgatctgaaaatctcgagccgagtgatcgattttgtccgtcttcgtctatagtccccggatgcacctcaattacagggtaaggagagacagggagttgatgtggaaatttcataggggataattttctattattgtaattgttctaacttcgtctctctaagcactgcatctagcctgcctgccaagggtggaactagtatgattacattaccctagtgatgttcttttgggagatattgctgctgttatattgtttgtccattaccatttctatgataatcaccgatcaagggcggatccagaaacaaaaagttggggggactcaaataccgagtacaccgatggtacc

SEQ ID NO: 12 - protein, 487 aa, Oryza sativa
MSVASPHHLRPSPLAPACRAGGVPARAAAAHRPWCPRVRRAVAAASSGGGGGEAVTAVSAAAVGAPASAARDTRGYNIESLAVGLNKDKAMFTIVVSGTDRVLNQVIEQLNKLVNVLNLEIPRAETFYPNLVKTLALFIENLLRVEDLSKEPQVERELMLIKINVEPDQRPEVMVLVDIFRAKVVDISENTLTIEVTGDPGKIVAVQRNLSKFGIKEICRTGKIALRREKIGATARFWGFSAASYPDLIEALPKNSLLTSVNKTVNGSFDQPSNAGGDVYPVEPYEGSSMNQVLDAHWGVLDDEDSSGLRSHTLSILVNDCPGVLNIVTGVFARRGYNIQSLAVGPAEKSGLSRITTVAPGTDESIEKLVQQLNKLVDVHEVQDITHLPFAERELMLIKVSVNTAARRDILDIAEIFRAKSVDVSDHTVTLQLTGDLDKMVALQRLLEPYGICEIICANTVTVLTHIVSKTHSPHVMDTFAEVNTCF

(19) United States
(12) Patent Application Publication
(10) Pub. No.: US 2007/0250946 A1
(43) Pub. Date: Oct. 25, 2007

(54) Monocot Ahass Sequences and Methods of Use

(76) Inventors: Robert Ascenzi, Cary, NC (US); Gregory Budziszewski, Research Triangle, NC (US); Genichi Kakefuda, Princeton Junction, NJ (US); Bijay Singh, Cary, NC (US)

Correspondence Address: CONNOLLY BOVE LODGE & HUTZ, LLP, P O BOX 2207, WILMINGTON, DE, 19899, US

(21) Appl. No.: 11/659,129

(22) PCT Filed: Aug. 4, 2005
(86) PCT No.: PCT/US05/27729

§ 371 Date: Mar. 20, 2007

Related U.S. Application Data
(60) Provisional application No. 60/598,671, filed on Aug. 4, 2004.

(51) Int. Cl. A01H 1/00; A01H 5/00; A01N 43/50; C07H 21/04; C07K 1/00; C12N 15/00; C12N 5/00

(52) U.S. Cl. 800/278; 435/320.1; 435/410; 504/275; 530/350; 536/23.2; 800/295

(57) ABSTRACT

Isolated polynucleotides that encode acetohydroxyacid synthase small subunit (AHASS) polypeptides, and the amino acid sequences encoding these polypeptides, are described. Expression cassettes and expression vectors comprising the polynucleotides of the invention, as well as plants and host cells transformed with the polynucleotides, expression cassettes, and expression vectors, are described. Methods of using the polynucleotides to enhance the resistance of plants to herbicides are also described.

### FIELD OF THE INVENTION

This invention relates to novel polynucleotides that encode the small subunit of the acetohydroxyacid synthase enzyme and that can be used to enhance the acetohydroxyacid synthase activity and the herbicide-tolerance of crop plants.

### BACKGROUND OF THE INVENTION

Acetohydroxyacid synthase (AHAS; EC 4.1.3.18, also known as acetolactate synthase or ALS), is the first enzyme that catalyzes the biochemical synthesis of the branched chain amino acids valine, leucine, and isoleucine (Singh, 1999, “Biosynthesis of valine, leucine and isoleucine,” in Plant Amino Acids, Singh, ed., Marcel Dekker Inc. New York, N.Y., pp. 227-247). AHAS is the site of action of four structurally diverse herbicide families including the sulfonylureas (LaRossa and Falco, 1984, Trends Biotechnol. 2:158-161), the imidazolinones (Shaver et al., 1984, Plant Physiol. 76:545-546), the triazolopyrimidines (Subramanian and Gerwick, 1989, “Inhibition of acetolactate synthase by triazolopyrimidines,” in Biocatalysis in Agricultural Biotechnology, Whitaker and Sonnet, eds., ACS Symposium Series, American Chemical Society, Washington, D.C., pp. 277-288), and the pyrimidyloxybenzoates (Subramanian et al., 1990, Plant Physiol. 94:239-244). Imidazolinone and sulfonylurea herbicides are widely used in modem agriculture due to their effectiveness at very low application rates and relative non-toxicity in animals. By inhibiting AHAS activity, these families of herbicides prevent further growth and development of susceptible plants including many weed species. Several examples of commercially available imidazolinone herbicides are PURSUIT® (imazethapyr), SCEPTER® (imazaquin), and ARSENAL® (imazapyr). Examples of sulfonylurea herbicides are chlorsulfuron, metsulfuron methyl, sulfometuron methyl, chlorimuron ethyl, thifensulfuron methyl, tribenuron methyl, bensulfuron methyl, nicosulfuron, ethametsulfuron methyl, rimsulfuron, triflusulfuron methyl, triasulfuron, primisulfuron methyl, cinosulfuron, amidosulfluon, fluzasulfuron, imazosulfuron, pyrazosulfuron ethyl, and halosulfuron.

Due to their high effectiveness and low-toxicity, imidazolinone herbicides are favored for application by spraying over the top of a wide area of vegetation. The ability to spray an herbicide over the top of a wide range of vegetation decreases the costs associated with plantation establishment and maintenance, and decreases the need for site preparation prior to use of such chemicals. Spraying over the top of a desired tolerant species also results in the ability to achieve maximum yield potential of the desired species due to the absence of competitive species. However, the ability to use such spray-over techniques is dependent upon the presence of imidazolinone-resistant species of the desired vegetation in the spray over area.

Among the major agricultural crops, some leguminous species such as soybean are naturally resistant to imidazolinone herbicides due to their ability to rapidly metabolize the herbicide compounds (Shaner and Robson, 1985, Weed Sci. 33:469-471). Other crops such as corn (Newhouse et al., 1992, Plant Physiol. 100:882886) and rice (Barrette et al., 1989, Crop Safeners for Herbicides, Academic Press, New York, pp. 195-220) are somewhat susceptible to imidazolinone herbicides. The differential sensitivity to the imidazolinone herbicides is dependent on the chemical nature of the particular herbicide and differential metabolism of the compound from a toxic to a non-toxic form in each plant (Shaner et al., 1984, Plant Physiol. 76:545-546; Brown et al., 1987, Pestic. Biochem. Physiol. 27:24-29). Other plant physiological differences such as absorption and translocation also play an important role in sensitivity (Shaner and Robson, 1985, Weed Sci. 33:469-471).

Crop cultivars resistant to imidazolinones, sulfonylureas, and triazolopyrimidines have been successfully produced using seed, microspore, pollen, and callus mutagenesis in Zea mays, Arabidopsis thaliana, Brassica napus, Glycine max, and Nicotiana tabacum (Sebastian et al., 1989, Crop Sci. 29:1403-1408; Swanson et al., 1989, Theor. Appl. Genet. 78:525-530; Newhouse et al., 1991, Theor. Appl. Genet. 83:65-70; Sathasivan et al., 1991, Plant Physiol. 97:1044-1050; Mourand et al., 1993, J. Heredity 84:91-96). In all cases, a single, partially dominant nuclear gene conferred resistance. Four imidazolinone resistant wheat plants were also previously isolated following seed mutagenesis of Triticum aestivum L. cv. Fidel (Newhouse et al., 1992, Plant Physiol. 100:882-886). Inheritance studies confirmed that a single, partially dominant gene conferred resistance. Based on allelic studies, the authors concluded that the mutations in the four identified lines were located at the same locus. One of the Fidel cultivar resistance genes was designated FS-4 (Newhouse et al., 1992, Plant Physiol. 100:882-886).

Plant resistance to imidazolinone herbicides has also been reported in a number of patents. U.S. Pat. Nos. 4,761,373, 5,331,107, 5,304,732, 6,211,438, 6,211,439 and 6,222,100 generally describe the use of an altered AHAS gene to elicit herbicide resistance in plants, and specifically disclose certain imidazolinone resistant corn lines. U.S. Pat. No. 5,013,659 discloses plants exhibiting herbicide resistance due to mutations in at least one amino acid in one or more conserved regions. The mutations described therein encode either cross-resistance for imidazolinones and sulfonylureas or sulfonylurea-specific resistance, but imidazolinone-specific resistance is not described. Additionally, U.S. Pat. No. 5,731,180 and U.S. Pat. No. 5,767,361 discuss an isolated gene having a single amino acid substitution in a wild-type monocot AHAS amino acid sequence that results in imidazolinone-specific resistance. In addition, rice plants that are resistant to herbicides that interfere with acetohydroxyacid synthase have been developed by mutation breeding and also by the selection of herbicide resistant plants from a pool of rice plants produced by anther culture (See, U.S. Pat. Nos. 5,545,822, 5,736,629, 5,773,703, 5,773,704, 5,952,553 and 6,274,796).

In plants, the AHAS enzyme is comprised of two subunits: a large subunit (catalytic role) and a small subunit (regulatory role) (Duggleby and Pang, 2000, J. Biochem. Mol. Biol. 33:1-36). The AHAS large subunit protein (termed AHASL) may be encoded by a single gene as in the case of Arabidopsis and rice or by multiple gene family members as in maize, canola, and cotton. Specific, single-nucleotide substitutions in AHASL confer upon the enzyme a degree of insensitivity to one or more classes of herbicides (Chang and Duggleby, 1998, Biochem J. 333:765-777).

Herbicide resistant AHASL genes have also been rationally designed. WO 96/33270, U.S. Pat. Nos. 5,853,973 and 5,928,937 disclose structure-based modeling methods for the preparation of AHAS variants, including those that exhibit selectively increased resistance to herbicides such as imidazolines and AHAS-inhibiting herbicides. Computer-based modeling of the three dimensional conformation of the AHAS-inhibitor complex predicts several amino acids in the proposed inhibitor binding pocket as sites where induced mutations would likely confer selective resistance to imidazolinones (Ott et al., 1996, J. Mol. Biol. 263:359-368). Wheat plants produced with some of these rationally designed mutations in the proposed binding sites of the AHAS enzyme have in fact exhibited specific resistance to a single class of herbicides (Ott et al, 1996, J. Mol. Biol. 263:359-368).

A great deal is known about the function of AHAS enzymes from studies in prokaryotic systems. These studies have shed light on the role of the AHAS small subunit (AHASS) protein. The prokaryotic AHAS enzymes exist as two distinct, but physically associated, protein subunits. In prokaryotes, the two polypeptides, a “large subunit” and a “small subunit,” are expressed from separate genes. Three major AHAS enzymes, designated I, II and III, all having large and small subunits, have been identified in enteric bacteria. In prokaryotes, the AHAS enzyme has been shown to be a regulatory enzyme in the branched amino acid biosynthetic pathway (Miflin, 1971, Arch. Biochm. Biophys. 146:542-550), and only the large subunit has been observed as having catalytic activity. From studies of AHAS enzymes from microbial systems, two roles have been described for the small subunit. One role is the allosteric feedback inhibition of the catalytic large subunit when in the presence of isoleucine, leucine, or valine or combinations thereof. The other role is the enhancement of the catalytic activity of the large subunit in the absence of isoleucine, leucine, or valine. The small subunit has also been shown to increase the stability of the active conformation of the large subunit (Weinstock et al., 1992, J. Bacteriol. 174:5560-5566). The expression of the small subunit can also increase the expression of the large subunit as seen for AHAS I from E. coli (Weinstock et al., 1992, J. Bacteriol. 174:5560-5566).

In vitro studies have demonstrated that the prokaryotic large subunit exhibits, in the absence of the small subunit, a basal level of AHAS activity and that this activity cannot be feedback-inhibited by the amino acids isoleucine, leucine, or valine. When the small subunit is added to the same reaction mixture containing the large subunit, the specific activity of the large subunit increases.

While the small subunit of AHAS is also known to occur in plants, less is known about its in vivo function. WO 98/37206 discloses the nucleotide sequence encoding an AHASS cDNA sequence from Nicotiana plumbaginifolia and the use of this sequence in screening herbicides, which inhibit the activity of AHAS holoenzyme. In addition, WO 98/37206 discloses a partial-length cDNA sequence for a maize AHASS protein. U.S. Pat. No. 6,348,643 discloses the nucleotide and amino acid sequences of a full-length AHASS protein from Arabidopsis thaliana. That patent further discloses the activation of both wild type and herbicide-resistant forms of the Arabidopsis AHASL protein by addition of the Arabidopsis AHASS protein. The activation was demonstrated by disclosing the ability of an Arabidopsis AHASS protein to increase the specific AHAS activity of both wild-type and herbicide-resistant forms of the AHASL protein. More recently, U.S. Patent Publication No. 2001/0044939 reported the beneficial effects of reconstituting a native plant AHASS protein with an AHASL protein that is not species-specific, as shown by the ability of the N. plumbaginifolia AHASS protein to increase the specific activity of an AHASL protein from another dicotyledonous plant, Arabidopsis thaliana.

### SUMMARY OF THE INVENTION

The present invention provides isolated polynucleotides that encode maize, rice, and wheat acetohydroxyacid synthase small subunit (AHASS) polypeptides, which are referred to herein as Zea mays AHAS small subunit subtype 1 paralog a (ZmAHASS1a), Oryza sativa AHAS small subunit subtype 1 (OsAHASS1), and Triticum aestivum AHAS small subunit subtype 1 (TaAHASS1X), respectively. The polynucleotides of the present invention comprise a nucleotide sequence selected from the group consisting of the nucleotide sequences set forth in SEQ ID NOS:1 and 3, and nucleotide sequences encoding the amino acid sequences set forth in SEQ ID NOS:2, 4, and 5, and fragments and variants of the nucleotide sequences that encode a polypeptide comprising AHASS activity.

In one embodiment, the polynucleotides of the present invention comprise consecutive nucleotides 275-1495 of SEQ ID NO:1 or consecutive nucleotides 342-1565 of SEQ ID NO:3. In another embodiment, the polynucleotides of the present invention have at least 80% sequence identity with the nucleotide sequences set forth in SEQ ID NO:1 or SEQ ID NO:3, or with consecutive nucleotides 275-1495 of SEQ ID NO:1 or consecutive nucleotides 342-1565 of SEQ ID NO:3, wherein such polynucleotides encode a polypeptide that has AHASS activity. The isolated polynucleotides of the present invention also encompass polynucleotides encoding the mature form of the AHASS polypeptides of the present invention. Such mature forms of the AHASS polypeptides lack the chloroplast transit peptide located at the N-terminal end.

The present invention also provides polynucleotide sequences comprising a rice AHASS promoter. One skilled in the art will recognize that this polynucleotide comprises a region of the rice genome upstream from the transcription start site of the rice AHASS gene which one can manipulate to generate a minimal-length promoter that can still function in plants. The rice genomic fragment comprising this promoter is set forth in SEQ ID NO:10.

The present invention further provides polynucleotide sequences comprising a rice AHASS terminator. One skilled in the art will recognize this polynucleotide comprises a region of the rice genome downstream from the translation stop codon of the rice AHASS gene, which one can manipulate to generate a minimal-length terminator that can still function in plants. The rice genomic fragment comprising this terminator is set forth in SEQ ID NO:11.

The present invention also provides expression cassettes for expressing the polynucleotides of the present invention in plants, plant cells, and other non-human host cells, that include, but are not limited to bacteria, fungal cells, and animals cells. The expression cassettes comprise a promoter expressible in the plant, plant cell, or other host cell of interest, operably linked to a polynucleotide of the present invention that encodes either a full-length AHASS polypeptide (i.e. including the chloroplast transit peptide) or a mature AHASS polypeptide (i.e. without the chloroplast transit peptide). If expression is desired in the plastids of plants or plant cells, the expression cassette can further comprise an operably linked chloroplast-targeting sequence that encodes a chloroplast transit peptide.

The present invention further provides plant expression vectors for expressing both a eukaryotic AHASL polypeptide and an AHASS polypeptide in a plant or a host cell of interest. In one embodiment, the plant expression vectors comprise a first polynucleotide construct and a second polynucleotide construct, wherein the first polynucleotide construct comprises a first promoter operably linked to a nucleotide sequence encoding a eukaryotic AHASL polypeptide, wherein the second polynucleotide construct comprises a second promoter operably linked to a nucleotide sequence encoding an AHASS polypeptide, and wherein the first and second promoters are capable of driving gene expression in a plant or host cell of interest. In one embodiment, the first and second polynucleotide constructs further comprise an operably linked chloroplast-targeting sequence. In another embodiment, the eukaryotic AHASL polypeptide is a plant AHASL polypeptide, and in some cases is an herbicide-tolerant AHASL polypeptide.

The present invention provides isolated polypeptides comprising the AHASS polypeptides. The isolated polypeptides comprise an amino acid sequence selected from the group consisting of the amino acid sequences set forth in SEQ ID NOS:2, 4, and 5, the amino acid sequences encoded by nucleotide sequences set forth in SEQ ID NOS:1 and 3, and fragments and variants of the amino acid sequences that encode a polypeptide comprising AHASS activity. Such fragments include, but are not limited to, mature forms of the AHASS polypeptides of the present invention, particularly an amino acid sequence selected from the group consisting of: amino acids 77-483 of the amino acid sequence set forth in SEQ ID NO:2, amino acids 74-481 of the amino acid sequence set forth in SEQ ID NO:4, amino acids 64-471 of the amino acid sequence set forth in SEQ ID NO:5, the amino acid sequence encoded by nucleotides 275-1495 of the nucleotide sequence set forth in SEQ ID NO:1, and the amino acid sequence encoded by nucleotides 342-1565 of the nucleotide sequence set forth in SEQ ID NO:3. The present invention also provides polypeptides having at least 81% sequence identity with the amino acid sequence set forth in SEQ ID NOS:2, 4, or 5, or at least 77% sequence identity with consecutive amino acids 64-471 of SEQ ID NO:5, wherein such polypeptides comprise AHASS activity.

The present invention further provides transgenic plants, seeds, and transgenic plant cells that comprise an AHASS polynucleotide of the present invention. In one embodiment, the AHASS polynucleotide is operably linked to a promoter that drives its expression in a plant cell. In another embodiment, the promoter is either a constitutive promoter or a tissue-preferred promoter. In another embodiment, the polynucleotide construct further comprises a chloroplast-targeting sequence operably linked to the AHASS polynucleotide. In one embodiment, the transgenic plant is a monocot plant selected from a group consisting of maize, wheat, rice, barley, rye, oats, triticale, millet, and sorghum. In another embodiment, the transgenic plant is a dicot plant selected from a group consisting of soybean, cotton, Brassica spp., tobacco, potato, sugar beet, alfalfa, sunflower, safflower, and peanut. Preferably, these transgenic plants, seeds, and plant cells comprising the AHASS polynucleotide of the present invention have AHAS activity and/or resistance to at least one herbicide that is increased as compared to a wild type variety of the plant.

The present invention provides methods for enhancing AHAS activity in a plant comprising transforming a plant with an AHASS polynucleotide of the present invention. In one embodiment, the AHASS polynucleotide is in an expression cassette comprising a promoter, operably linked to the AHASS nucleotide sequence, that is capable of driving gene expression in a plant cell. In another embodiment, the promoter is either a constitutive promoter or a tissue-preferred promoter. In yet another embodiment, the plant comprises an herbicide-tolerant acetohydroxyacid synthase large subunit (AHASL) polypeptide. The present invention methods may be used to enhance or increase the resistance of a plant to at least one herbicide that interferes with the catalytic activity of the AHAS enzyme. A transgenic plant produced by these methods is also provided, wherein the AHAS activity in such a transgenic plant is increased as compared to a wild-type variety of the plant.

The present invention also provides methods for enhancing herbicide-tolerance in an herbicide-tolerant plant comprising transforming the plant with an AHASS polynucleotide of the present invention. In one embodiment, the AHASS polynucleotide is in an expression cassette comprising a promoter, operably linked to the AHASS nucleotide sequence, that is capable of driving gene expression in a plant cell. In another embodiment, the promoter is either a constitutive promoter or a tissue-preferred promoter. In one embodiment, the AHASS polynucleotide construct further comprises a nucleotide sequence encoding an herbicide-tolerant AHASL polypeptide. In another embodiment, the herbicide-tolerant plant comprises an AHASL polypeptide. In yet another embodiment, the herbicide-tolerant plant is or is not genetically engineered to express the herbicide-tolerant AHASL polypeptide. In another embodiment, the herbicide-tolerant plant is an imidazolinone-tolerant plant. A transgenic plant produced by these methods is also provided, wherein the AHAS activity in such a transgenic plant is increased as compared to a wild-type variety of the plant. The invention also provides methods for controlling weeds in the vicinity of a plant, comprising applying an imidazolinone herbicide to the weeds and to the plant, wherein the plant has increased tolerance to the imidazolinone herbicide as compared to a wild type variety of the plant and wherein the plant comprises a polynucleotide construct that comprises an AHASS nucleotide sequence of the present invention. In one embodiment, the AHASS nucleotide sequence is defined in SEQ ID NO:1, SEQ ID NO:3; consecutive nucleotides 275-1495 of SEQ ID NO:1, or consecutive nucleotides 342-1565 of SEQ ID NO:3. In another embodiment, the AHASS nucleotide comprises a polynucleotide encoding a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5.

The present invention further provides isolated fusion polypeptides comprising an AHASL domain operably linked to an AHASS domain, wherein the fusion polypeptide comprises AHAS activity. The AHASL domain comprises an amino acid sequence of a mature eukaryotic AHASL polypeptide. The AHASS domain comprises an amino acid sequence selected from the group consisting of the amino acid sequences set forth in SEQ ID NOS:2, 4, and 5; the amino acid sequences encoded by nucleotide sequences set forth in SEQ ID NOS:1 and 3; and fragments and variants of the amino acid sequences that encode a polypeptide comprising AHASS activity. Such fragments include, but are not limited to, mature forms of the AHASS polypeptides of the present invention, particularly an amino acid sequence selected from the group consisting of: amino acids 77-483 of the amino acid sequence set forth in SEQ ID NO:2, amino acids 74-481 of the amino acid sequence set forth in SEQ ID NO:4, amino acids 64-471 of the amino acid sequence set forth in SEQ ID NO:5, the amino acid sequences encoded by nucleotides 275-1495 of the nucleotide sequence set forth in SEQ ID NO:1, and nucleotides 342-1565 of the nucleotide sequence set forth in SEQ ID NO:3. In one embodiment, the eukaryotic AHASL polypeptide is a plant AHASL polypeptide. In another embodiment, the eukaryotic AHASL polypeptide is an herbicide-tolerant plant AHASL polypeptide. In yet another embodiment, the fusion polypeptide further comprises a linker region operably linked between the AHASL domain and the AHASS domain. Preferably, the AHASL polypeptide and the AHASS polypeptide are from different species.

The present invention also provides expression vectors for expressing an AHASL-AHASS fusion polypeptide in a plant or host cell of interest. The expression vector comprises a promoter operably linked to a polynucleotide encoding an AHASL-AHASS fusion polypeptide. The polynucleotide comprises a first nucleotide sequence operably linked to a second nucleotide sequence, wherein the first nucleotide sequence encodes an amino acid sequence comprising a eukaryotic mature AHASL polypeptide and the second nucleotide sequence encodes an amino acid sequence comprising a mature AHASS polypeptide of the present invention. The polynucleotide may further comprise an operably linked third nucleotide sequence encoding a linker region, which is situated between the AHASL and AHASS domains of the fusion polypeptide. In one embodiment, the polynucleotide encoding an AHASL-AHASS fusion polypeptide further comprises an operably linked chloroplast-targeting sequence. In another embodiment, the eukaryotic AHASL domain of the fusion polypeptide is a plant AHASL polypeptide. In yet another embodiment, the eukaryotic AHASL polypeptide is an herbicide-tolerant plant AHASL polypeptide.

The present invention further provides transgenic plants, seeds, and plant cells comprising a polynucleotide encoding an AHASL-AHASS fusion polypeptide. Also provided are methods for producing an herbicide-tolerant plant, comprising transforming a plant cell with an expression vector comprising a promoter operably linked to a polynucleotide encoding an AHASL-AHASS fusion polypeptide, and generating a transgenic plant from the transgenic plant cell, wherein the transgenic plant comprising the AHASL-AHASS fusion polypeptide has increased tolerance to at least one herbicide as compared to a wild type variety of the plant.

### BRIEF DESCRIPTION OF THE DRAWINGS

FIG. 1 is an amino acid sequence alignment of the mature AHASS polypeptides of the present invention: ZmAHASS1a (residues 77-483 of SEQ ID NO:2), OsAHASS1 (residues 74-481 of SEQ ID NO:4), and TaAHASS1X (residues 64-471 of SEQ ID NO:5). The deduced amino acid sequences (minus the predicted variable chloroplast transit peptide) above were aligned using the Clustal X version 1.81, Multiple Alignment Mode. Complete alignment was performed iteratively (at least three times) using the default parameters. “*” indicates that the amino acid is identical in all sequences. “:” and “.” are decreasingly conservative substitutions. The conserved Domain 1 and Domain 2 regions are indicated in bold. Domain 1 is at the N-terminus, and Domain 2 is at the C-terminus. There is an intervening, variable linker region that is situated between Domains 1 and 2.

FIG. 2 provides percent amino acid sequence identities from pairwise comparisons of mature AHASS polypeptides. The comparisons include all publicly known plant AHASS sequences and the amino acid sequences of the present invention for ZmAHASS1a (SEQ ID NO:2), OsAHASS1 (SEQ ID NO:4), and TaAHASS1X (SEQ ID NO:5). The deduced amino acid sequences from the coding sequences of all published genes and other putative full-length sequences were aligned using the ClustalW algorithm. Pairwise differences were calculated based on this alignment. The data are presented in the format of percent sequence identity between two sequences. Nomenclature: “GmAHASS1” refers to Glycine max AHAS small subunit subtype 1 (SEQ ID NO:18 of U.S. Patent Application Publication No. 2001/00044039A1); “NpAHASS1” refers to Nicotiana plumbaginifolia AHAS small subunit subtype 1 (Accession No. AJ234901.1); “ZmAHASS2” refers to Zea mays AHAS small subunit subtype 2 (SEQ ID NO:10 of U.S. Patent Application Publication No. 2001/00044039A1); “OsAHASS2” refers to Oryza sativa AHAS small subunit subtype 2 (SEQ ID NO:16 of U.S. Patent Application Publication No. 2001/00044039A1); “AtAHASS1” refers to Arabidopsis thaliana AHAS small subunit subtype 1 (NM_{—}179843.1); and “AtAHASS2” refers to A. thaliana AHAS small subunit subtype 2 (NM_{—}121634.2).

FIG. 3 provides percent amino acid sequence identities from pairwise comparisons of Domain 1 of AHASS polypeptides. The comparisons include Domain 1 from all publicly known plant AHASS sequences and from the amino acid sequences of the present invention for ZmAHASS1a (SEQ ID NO:2), OsAHASS1 (SEQ ID NO:4), and TaAHASS1X (SEQ ID NO:5). The nomenclature for the amino acid sequences and the percent amino acid sequence identities are as described above for FIG. 2 except that only the amino acid sequence corresponding to Domain 1 was used in determining percent sequence identity.

FIG. 4 provides percent amino acid sequence identities from pairwise comparisons of Domain 2 of AHASS polypeptides. The comparisons include Domain 2 from all publicly known plant AHASS sequences and from the amino acid sequences of the present invention for ZmAHASS1a (SEQ ID NO:2), OsAHASS1 (SEQ ID NO:4), and TaAHASS1X (SEQ ID NO:5). The nomenclature for the amino acid sequences and the percent amino acid sequence identities are as described above for FIG. 2 except that only the amino acid sequence corresponding to Domain 2 was used in determining percent sequence identity. Domains 1 and 2 were empirically determined from the amino acid sequences of known AHASS polypeptides. Each domain contains an ACT domain. The known plant AHASS polypeptides have two repeats of a bacteria-like AHASS polypeptide. Despite the likelihood of being the result of an ancient duplication, the “repeats” are now quite distinct from each other and are referred to herein as Domains 1 and 2.

FIG. 5 provides an alignment of the amino acid sequences of OsAHASS1 (SEQ ID NO:4) and a translation of annotations of the OsAHASS1 genomic DNA that are available from The Institute for Genomic Research (TIGR) (SEQ ID NO:12). Amino acids that are identical at the corresponding positions in the two amino acid sequences are shaded. A consensus sequence is also provided.

FIG. 6 depicts the alignment and regions of overlap of two ESTs and one proprietary contig used to construct the full-length OsAHASS1 nucleotide sequence (SEQ ID NO:3).

### DETAILED DESCRIPTION OF THE INVENTION

The present invention relates to isolated polynucleotide molecules comprising nucleotide sequences that encode acetohydroxyacid synthase small subunit (AHASS) polypeptides. Specifically, the present invention relates to isolated polynucleotide molecules that encode monocot AHASS polypeptides from maize (Zea mays), rice (Oryza sativa), and wheat (Triticum aestivum), which are referred to herein as ZmAHASS1a, OsAHASS1, and TaAHASS1X, respectively. More specifically, the present invention relates to isolated polynucleotide molecules comprising a polynucleotide sequence selected from the group consisting of: a nucleotide sequence as defined in SEQ ID NO:1 or SEQ ID NO:3, a nucleotide sequence encoding an AHASS polypeptide as defined in SEQ ID NOS:2, 4, and 5, and fragments and variants of such nucleotide sequences that encode functional AHASS polypeptides.

In addition, the present invention provides isolated polynucleotides encoding a mature ZmAHASS1a, OsAHASS1, or TaAHASS1X polypeptide. The mature AHASS polypeptides of the present invention lack the chloroplast transit peptide that is found at the N-terminal end of each of the ZmAHASS1a, OsAHASS1, and TaAHASS1X polypeptides, but retain AHASS activity. In particular, the polynucleotides of the present invention comprise a nucleotide sequence selected from the group consisting of: nucleotides 275-1495 of the nucleotide sequence set forth in SEQ ID NO:1, nucleotides 342-1565 of the nucleotide sequence set forth in SEQ ID NO:3, a nucleotide sequence encoding amino acids 77-483 of the amino acid sequence set forth in SEQ ID NO:2, a nucleotide sequence encoding amino acids 64-471 of the amino acid sequence set forth in SEQ ID NO:4, a nucleotide sequence encoding amino acids 74-481 of the amino acid sequence set forth in SEQ ID NO:5, and fragments and variants of these nucleotide sequences that encode a mature AHASS polypeptide comprising AHASS activity.

As used herein unless otherwise indicated, “AHASS activity” refers to a biological activity of an AHASS polypeptide, whereby the AHASS polypeptide increases the AHAS activity of at least one AHASL polypeptide when such AHASS and AHASL polypeptides are in the presence of each other, as compared to the AHAS activity of the AHASL polypeptide in the absence of the AHASS polypeptide.

The isolated AHASS polynucleotide molecules of the present invention can be used to transform crop plants to enhance the tolerance of the crop plants to herbicides, particularly herbicides that are known to inhibit AHAS activity, and in particular, imidazolinone and sulfonylurea herbicides. Such AHASS polynucleotide molecules can be used in expression cassettes, expression vectors, transformation vectors, plasmids, and the like. The transgenic plants obtained following transformation with such polynucleotide constructs show increased tolerance to AHAS-inhibiting herbicides such as, for example, imidazolinone and sulfonylurea herbicides. As used herein, the terms “tolerance” and “resistance” are used interchangeably and refer to the ability of a plant to withstand the effect of an herbicide at a level that would normally kill, or inhibit the growth of, a wild-type variety of the plant. As used herein, a “wild-type variety” of the plant refers to a group of plants that are analyzed for comparative purposes as a control plant, wherein the wild type variety of the plant is identical to the test plant (plant transformed with an AHASS polynucleotide or plant in which expression of the AHASS polypeptide has been modified) with the exception that the wild type variety of the plant has not been transformed with an AHASS polynucleotide and/or expression of the AHASS polynucleotide in the wild type variety plant has not been modified. The use of the term “wild-type variety” plant, therefore, is not intended to imply that the plant lacks recombinant DNA in its genome.

Compositions of the present invention include nucleotide sequences that encode AHASS polypeptides. In particular, the present invention provides for isolated polynucleotide molecules (also referred to herein as “nucleic acid molecules”) comprising nucleotide sequences encoding the amino acid sequences shown in SEQ ID NOS:2, 4, and 5. Further provided are polypeptides having an amino acid sequence encoded by a polynucleotide molecule described herein, for example, those set forth in SEQ ID NOS:1 and 3, and fragments and variants thereof.

The present invention encompasses isolated or substantially purified nucleic acid or polypeptide compositions. An “isolated” or “purified” polynucleotide molecule or polypeptide, or biologically active portion thereof, is substantially or essentially free from components that normally accompany or interact with the polynucleotide molecule or polypeptide as found in its naturally occurring environment. Thus, an isolated or purified polynucleotide molecule or polypeptide is substantially free of other cellular material, or culture medium when produced by recombinant techniques, or substantially free of chemical precursors or other chemicals when chemically synthesized. Preferably, an “isolated” nucleic acid is free of sequences (preferably polypeptide encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated polynucleotide molecule can contain less than about 5 kb, 4 kb, 3 kb, 2 kb, 1 kb, 0.5 kb, or 0.1 kb of nucleotide sequences that naturally flank the polynucleotide molecule in genomic DNA of the cell from which the nucleic acid is derived. A polypeptide that is substantially free of cellular material includes preparations of polypeptide having less than about 30%, 20%, 10%, 5%, or 1% (by dry weight) of contaminating polypeptide. When the polypeptide of the present invention or biologically active portion thereof is recombinantly produced, preferably culture medium represents less than about 30%, 20%, 10%, 5%, or 1% (by dry weight) of chemical precursors or non-polypeptide-of-interest chemicals.

The present invention provides isolated polypeptides comprising the AHASS polypeptides: ZmAHASS1a, OsAHASS1, and TaAHASS1X. As used herein, the terms “protein” and “polypeptide” are used interchangeably to refer to a chain of at least four amino acids joined by peptide bonds. The chain may be linear, branched, circular, or combinations thereof. The isolated polypeptides may comprise an amino acid sequence selected from the group consisting of the amino acid sequences set forth in SEQ ID NOS:2, 4, and 5; the amino acid sequences encoded by nucleotide sequences set forth in SEQ ID NOS:1 and 3; and functional fragments and variants of the amino acid sequences that encode an AHASS polypeptide comprising AHASS activity. The term “functional fragments and variants” refers to fragments and variants of the exemplified polypeptides that comprise AHASS activity.

Additionally provided are isolated polypeptides comprising the mature forms of the AHASS polypeptides of the present invention. Such isolated polypeptides comprise an amino acid sequence selected from the group consisting of: amino acids 77-483 of the amino acid sequence set forth in SEQ ID NO:2, amino acids 74-481 of the amino acid sequence set forth in SEQ ID NO:4, amino acids 64-471 of the amino acid sequence set forth in SEQ ID NO:5, the amino acid sequence encoded by nucleotides 275-1495 of the nucleotide sequence set forth in SEQ ID NO:1, the amino acid sequence encoded by nucleotides 342-1565 of the nucleotide sequence set forth in SEQ ID NO:3, and fragments and variants of the amino acid sequences that encode a mature AHASS polypeptide comprising AHASS activity.

In certain embodiments of the present invention, the methods involve the use of herbicide-tolerant or herbicide-resistant plants. An “herbicide-tolerant” or “herbicide-resistant” plant refers to a plant that is tolerant or resistant to at least one herbicide at a level that would normally kill, or inhibit the growth of, a normal or wild-type variety of the plant. Preferably, the herbicide-tolerant plants of the present invention comprise an herbicide-tolerant or herbicide-resistant AHASL protein. The term “herbicide-tolerant AHASL protein” or “herbicide-resistant AHASL protein” refers to an AHASL protein that displays higher AHAS activity, as compared to the AHAS activity of a wild-type AHASL protein, when in the presence of an herbicide that is known to interfere with AHAS activity and at a concentration or level that is to known to inhibit the AHAS activity of the wild-type AHASL protein.

Further, it is recognized that an herbicide-tolerant or herbicide-resistant AHASL protein can be introduced into a plant by transforming a plant or ancestor thereof with a nucleotide sequence encoding an herbicide-tolerant or herbicide-resistant AHASL protein. Such herbicide-tolerant or herbicide-resistant AHASL proteins are encoded by the herbicide-tolerant or herbicide-resistant AHASL polynucleotides. Alternatively, an herbicide-tolerant or herbicide-resistant AHASL protein may occur in a plant as a result of a naturally occurring or induced mutation in an endogenous AHASL gene in the genome of a plant or ancestor thereof.

The present invention provides transformed plants, transformed plant tissues, transformed plant cells, and transformed host cells with increased resistance or tolerance to at least one herbicide. The preferred amount or concentration of the herbicide is an “effective amount” or “effective concentration.” The term “effective amount” or “effective concentration” refers to an amount or concentration that is sufficient to kill or inhibit the growth of a similar, untransformed, plant, plant tissue, plant cell, or host cell, but that the amount does not kill or inhibit as severely the growth of the transformed plants, transformed plant cells, or transformed host cells. The term “similar, untransformed, plant, plant cell or host cell” refers to a plant, plant tissue, plant cell, or host cell, respectively, that lacks the particular polynucleotide of the present invention that was used to make the transformed plant, transformed plant cell, or transformed host cell of the present invention. The use of the term “untransformed” is not, therefore, intended to imply that a plant, plant tissue, plant cell, or other host cell lacks recombinant DNA in its genome.

The present invention provides methods for enhancing the tolerance or resistance of a plant, plant tissue, plant cell, or other host cell to at least one herbicide that interferes with the activity of the AHAS enzyme. Preferably, such an herbicide is an imidazolinone or sulfonylurea herbicide. For the present invention, the imidazolinone herbicides include, but are not limited to, PURSUIT® (imazethapyr), CADRE® (imazapic), RAPTOR® (imazamox), SCEPTER® (imazaquin), ASSERT® (imazethabenz), ARSENAL® (imazapyr), a derivative of any of the aforementioned herbicides, or a mixture of two or more of the aforementioned herbicides, for example, imazapyr/imazamox (ODYSSEY®). More specifically, the imidazolinone herbicide can be selected from, but is not limited to, 2-(4-isopropyl-4-methyl-5-oxo-2-imidiazolin-2-yl)-nicotinic acid, [2-(4-isopropyl)-4-] [methyl-5-oxo-2-imidazolin-2-yl)-3-quinolinecarboxylic] acid, [5-ethyl-2-(4-isopropyl-] 4-methyl-5-oxo-2-imidazolin-2-yl)-nicotinic acid, 2-(4-isopropyl-4-methyl-5-oxo-2-imidazolin-2-yl)-5-(methoxymethyl)-nicotinic acid, [2-(4-isopropyl-4-methyl-5-oxo-2-] imidazolin-2-yl)-5-methylnicotinic acid, and a mixture of methyl [6-(4-isopropyl-4-] methyl-5-oxo-2-imidazolin-2-yl)-m-toluate and methyl [2-(4-isopropyl-4-methyl-5-] oxo-2-imidazolin-2-yl)-p-toluate. The use of 5-ethyl-2-(4-isopropyl-4-methyl-5-oxo-2-imidazolin-2-yl)-nicotinic acid and [2-(4-isopropyl-4-methyl-5-oxo-2-imidazolin-2-] yl)-5-(methoxymethyl)-nicotinic acid is preferred. The use of [2-(4-isopropyl-4-] methyl-5-oxo-2-imidazolin-2-yl)-5-(methoxymethyl)-nicotinic acid is particularly preferred. For the present invention, the sulfonylurea herbicides include, but are not limited to, chlorsulfuron, metsulfuron methyl, sulfometuron methyl, chlorimuron ethyl, thifensulfuron methyl, tribenuron methyl, bensulfuron methyl, nicosulfuron, ethametsulfuron methyl, rimsulfuron, triflusulfuron methyl, triasulfuron, primisulfuron methyl, cinosulfuron, amidosulfluon, fluzasulfuron, imazosulfuron, pyrazosulfuron ethyl, and halosulfuron.

The present invention provides methods for enhancing AHAS activity in a plant comprising transforming a plant with an AHASS polynucleotide construct. As used herein, the term “AHASS polynucleotide construct” refers to a polynucleotide that comprises an AHASS nucleotide sequence. The methods comprise introducing a polynucleotide construct of the present invention into at least one plant cell and generating a transformed plant therefrom. In one embodiment, the AHASS polynucleotide construct comprises a promoter operably linked to the AHASS nucleotide sequence, wherein the promoter is capable of driving gene expression in a plant cell. Preferably, such a promoter is a constitutive promoter or a tissue-preferred promoter. The methods may be used to enhance or increase the tolerance of a plant to at least one herbicide that interferes with the catalytic activity of the AHAS enzyme.

The present invention also provides methods for enhancing herbicide-tolerance in an herbicide-tolerant plant, comprising transforming the plant with an AHASS polynucleotide construct. These methods comprise introducing an AHASS polynucleotide construct of the present invention into at least one plant cell and regenerating a transformed plant therefrom. In one embodiment, the herbicide-tolerant plant comprises an herbicide-tolerant AHASL protein that confers on the plant tolerance to at least one herbicide that is known to interfere with the activity of the AHAS enzyme. In another embodiment, the AHASS polynucleotide construct comprises a promoter operably linked to the AHASS nucleotide sequence, wherein the promoter is capable of driving gene expression in a plant cell. The methods may be used to increase the tolerance of an herbicide-tolerant plant to at least one herbicide that interferes with the activity of the AHAS enzyme. Thus, the methods allow for the application of higher levels of an herbicide to an herbicide-tolerant plant without killing or significantly injuring the herbicide-tolerant plant.

The present invention provides expression cassettes for expressing the AHASS polynucleotides of the present invention in plants, plant tissues, plant cells, and other host cells. The expression cassettes comprise a promoter expressible in the plant, plant tissue, plant cell, or other host cell of interest operably linked to a polynucleotide of the present invention that encodes either a full-length AHASS polypeptide (i.e. including the chloroplast transit peptide) or a mature AHASS polypeptide (i.e. without the chloroplast transit peptide). If expression is desired in the plastids of plants or plant cells, the expression cassette may comprise an operably linked chloroplast-targeting sequence that encodes a chloroplast transit peptide.

The expression cassettes of the present invention may be used in methods for enhancing the herbicide tolerance of a plant or a host cell. The methods involve transforming the plant or host cell with an expression cassette of the present invention, wherein the expression cassette comprises a promoter that is expressible in the plant or host cell of interest and wherein the promoter is operably linked to an AHASS polynucleotide of the present invention.

The present invention also provides expression vectors for expressing in a plant or a host cell of interest a eukaryotic AHASL polypeptide and an AHASS polypeptide of the present invention. In one embodiment, the plant expression vectors comprise a first polynucleotide construct and a second polynucleotide construct, wherein the first polynucleotide construct comprises a first promoter operably linked to a nucleotide sequence encoding a eukaryotic AHASL protein, wherein the second polynucleotide construct comprises a second promoter operably linked to a nucleotide sequence encoding an AHASS protein, and wherein the first and second promoters are capable of driving gene expression in a plant or host cell of interest. In one embodiment, the first and second polynucleotide constructs further comprise an operably linked chloroplast-targeting sequence. In another embodiment, the eukaryotic AHASL protein is a plant AHASL protein, and in some cases is an herbicide-tolerant AHASL protein. For expression in plants and plant cells, the expression vector is referred to herein as a plant expression vector. The first and second promoters of a plant expression vector are capable of driving gene expression in a plant cell.

The use of the term “polynucleotide constructs” herein is not intended to limit the present invention to polynucleotide constructs comprising DNA. Those of ordinary skill in the art will recognize that polynucleotide constructs, particularly polynucleotides and oligonucleotides, comprised of ribonucleotides and combinations of ribonucleotides and deoxyribonucleotides may also be employed in the methods disclosed herein. Thus, the polynucleotide constructs of the present invention encompass all polynucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The polynucleotide constructs of the present invention also encompass all forms of polynucleotide constructs including, but not limited to, single-stranded forms, double-stranded forms, hairpins, stem-and-loop structures, and the like. Furthermore, it is understood by those of ordinary skill in the art that each nucleotide sequence disclosed herein also encompasses the complement of that exemplified nucleotide sequence.

Furthermore, it is recognized that the methods of the present invention may employ a polynucleotide construct that is capable of directing, in a transformed plant, the expression of at least one protein, or at least one RNA, such as, for example, an antisense RNA that is complementary to at least a portion of an mRNA. Typically such a polynucleotide construct is comprised of a coding sequence for a protein or an RNA operably linked to 5′ and 3′ transcriptional regulatory regions. Alternatively, it is also recognized that the methods of the present invention may employ a polynucleotide construct that is not capable of directing, in a transformed plant, the expression of a protein or an RNA.

The present invention provides fusion proteins comprising a eukaryotic AHASL domain operably linked to an AHASS domain, wherein the AHASL domain comprises an amino acid sequence of a mature eukaryotic AHASL protein, and wherein the AHASS domain comprises an amino acid sequence of an AHASS protein of the present invention. The AHASL domain may comprise an amino acid sequence of a mature eukaryotic AHASL protein that is from the same or a different eukaryotic species as the amino acid sequence of the AHASS protein of the AHASS domain. Thus, the AHASL domain comprises any known amino acid sequence of a mature eukaryotic AHASL protein from any eukaryotic organism including, but not limited to, a monocotyledonous plant, a dicotyledonous plant, an alga, an animal, or a fungus. The present invention also provides nucleotide sequences encoding such fusion proteins.

The present invention provides expression vectors for expressing an AHASL-AHASS fusion polypeptide in a plant or a host cell of interest. The expression vector comprises a promoter, capable of driving gene expression in the plant or host cell of interest, operably linked to a polynucleotide encoding an AHASL-AHASS fusion polypeptide. The polynucleotide comprises a first nucleotide sequence that encodes an amino acid sequence comprising a eukaryotic mature AHASL polypeptide and is operably linked to a second nucleotide sequence that encodes an amino acid sequence comprising a mature AHASS polypeptide of the present invention. In particular embodiments, the polynucleotide further comprises an operably linked third nucleotide sequence encoding a linker region that is situated between the first and second nucleotide sequences.

When expressed in a plant or host cell, the AHASL-AHASS fusion polypeptides of the present invention comprise AHAS activity. Preferably, an AHASL-AHASS fusion polypeptide comprises a level of AHAS activity that is higher than the activity of the corresponding AHASL polypeptide when in the absence of the corresponding AHASS polypeptide.

The present invention provides methods for producing an herbicide-tolerant plant, comprising transforming a plant cell with a plant expression vector comprising a promoter operably linked to a polynucleotide encoding an AHASL-AHASS fusion polypeptide and generating a transgenic plant from the transgenic plant cell. The methods may be used to produce crop plants with increased tolerance to at least one herbicide that interferes with the AHAS enzyme.

The present invention encompasses host cells transformed with the polynucleotides described herein including, but not limited to, AHASS nucleotide sequences, nucleotide sequences encoding AHASL-AHASS fusion polypeptides, polynucleotide constructs, expression cassettes, and expression vectors. The host cells of the present invention encompass both prokaryotic and eukaryotic cells, including, but not limited to, plant cells, animal cells, bacterial cells, yeast cells, and other fungal cells. Preferably, the host cells of the present invention are non-human host cells. More preferably, the host cells are plant cells, bacterial cells, and yeast cells. Most preferably, the host cells are plant cells.

Further, it is recognized that, for expression of a polynucleotide of the present invention in a host cell of interest, the polynucleotide may be operably linked to a promoter that is capable of driving gene expression in the host cell of interest. The methods of the present invention for expressing the polynucleotides in host cells do not depend on a particular promoter. The methods encompass the use of any promoter that is known in the art and that is capable of driving gene expression in the host cell of interest.

The present invention encompasses AHASS polynucleotide molecules and fragments and variants thereof. Polynucleotide molecules that are fragments of these nucleotide sequences are also encompassed by the present invention. The term “fragment” refers to a portion of the nucleotide sequence encoding an AHASS polypeptide of the present invention. A fragment of an AHASS nucleotide sequence of the present invention may encode a biologically active portion of an AHASS polypeptide, or it may be a fragment that can be used as a hybridization probe or PCR primer using methods disclosed below. A biologically active portion of an AHASS polypeptide can be prepared by isolating a portion of one of the AHASS nucleotide sequences of the present invention, expressing the encoded portion of the AHASS polypeptide (e.g., by recombinant expression in vitro), and assessing the activity of the encoded portion of the AHASS polypeptide. Polynucleotide molecules that are fragments of an AHASS nucleotide sequence comprise at least about 15, 20, 50, 75, 100, 200, 300, 350, 400, 450, 500, 550, 600, 650, 700, 750, 800, 850, 900, 950, 1000, 1050, 1100, 1150, 1200, 1250, 1300, 1350, 1400, 1500, 1600, 1700, or 1800 nucleotides, or up to the number of nucleotides present in a full-length nucleotide sequence disclosed herein (for example, 1726 and 1861 nucleotides for SEQ ID NOS:1 and 3, respectively) depending upon the intended use.

It is understood that isolated fragments include any contiguous sequence not disclosed prior to the present invention as well as sequences that are substantially the same and which are not disclosed. Accordingly, if an isolated fragment is disclosed prior to the present invention, that fragment is not intended to be encompassed by the present invention. When a sequence is not disclosed prior to the present invention, an isolated nucleic acid fragment is at least about 12, 15, 20, 25, or 30 contiguous nucleotides. Other regions of the nucleotide sequence may comprise fragments of various sizes, depending upon potential homology with previously disclosed sequences.

A fragment of an AHASS nucleotide sequence that encodes a biologically active portion of an AHASS polypeptide of the present invention will encode at least about 15, 25, 30, 50, 75, 100, 125, 150, 175, 200, 250, 300, 350, 400, or 450 contiguous amino acids, or up to the total number of amino acids present in a full-length AHASS polypeptide of the present invention (for example, 483, 481, and 471 amino acids for SEQ ID NOS:2, 4, and 5, respectively). Fragments of an AHASS nucleotide sequence that are useful as hybridization probes for PCR primers generally need not encode a biologically active portion of an AHASS polypeptide.

Polynucleotide molecules that are variants of the nucleotide sequences disclosed herein are also encompassed by the present invention. “Variants” of the AHASS nucleotide sequences of the present invention include those sequences that encode the AHASS polypeptides disclosed herein but that differ conservatively because of the degeneracy of the genetic code. These naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the AHASS polypeptide disclosed in the present invention as discussed below. Generally, nucleotide sequence variants of the present invention will have at least about 75%, 80%, 85%, 90%, 91%, 92%, 93%, 94%, 95%, 96%, 97%, 98%, or 99% identity to a particular nucleotide sequence disclosed herein. A variant AHASS nucleotide sequence will encode an AHASS polypeptide, respectively, that has an amino acid sequence having at least about 75%, 80%, 85%, 90%, 91%, 92%, 93%, 94%, 95%, 96%, 97%, 98%, or 99% identity to the amino acid sequence of an AHASS polypeptide disclosed herein.

In addition, the skilled artisan will further appreciate that changes can be introduced by mutation into the nucleotide sequences of the present invention thereby leading to changes in the amino acid sequence of the encoded AHASS polypeptides without altering the biological activity of the AHASS polypeptides. Thus, an isolated polynucleotide molecule encoding an AHASS polypeptide having a sequence that differs from that of SEQ ID NOS:2, 4, or 5, respectively, can be created by introducing one or more nucleotide substitutions, additions, or deletions into the corresponding nucleotide sequence disclosed herein, such that one or more amino acid substitutions, additions, or deletions are introduced into the encoded polypeptide. Mutations can be introduced by standard techniques, such as site-directed mutagenesis and PCR-mediated mutagenesis. Such variant nucleotide sequences are also encompassed by the present invention.

For example, preferably, conservative amino acid substitutions may be made at one or more predicted, preferably nonessential amino acid residues. A “nonessential” amino acid residue is a residue that can be altered from the wild-type sequence of an AHASS polypeptide (e.g., the sequence of SEQ ID NOS:2, 4, or 5, respectively) without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Such substitutions would not be made for conserved amino acid residues, or for amino acid residues residing within a conserved motif.

The proteins of the present invention may be altered in various ways including amino acid substitutions, deletions, truncations, and insertions. Methods for such manipulations are generally known in the art. For example, amino acid sequence variants of the AHASS polypeptides can be prepared by making mutations in the DNA. Methods for mutagenesis and nucleotide sequence alterations are well known in the art. See, for example, Kunkel, 1985, Proc. Natl. Acad. Sci. USA 82:488-492; Kunkel et al., 1987, Methods in Enzymol. 154:367-382; U.S. Pat. No. 4,873,192; Walker and Gaastra, eds., 1983, Techniques in Molecular Biology, MacMillan Publishing Company, New York, and the references cited therein. Guidance as to appropriate amino acid substitutions that do not affect biological activity of the protein of interest may be found in the model of Dayhoff et al., 1978, Atlas of Protein Sequence and Structure, Natl. Biomed. Res. Found., Washington, D.C., herein incorporated by reference. Conservative substitutions, such as exchanging one amino acid with another having similar properties, may be preferable.

Alternatively, variant AHASS nucleotide sequences can be made by introducing mutations randomly along all or part of an AHASS coding sequence, such as by saturation mutagenesis, and the resultant mutants can be screened for AHASS biological activity to identify mutants that retain activity. Following mutagenesis, the encoded polypeptide can be expressed recombinantly, and the activity of the polypeptide can be determined using standard assay techniques.

Thus, the nucleotide sequences of the present invention include the sequences disclosed herein as well as fragments and variants thereof. The AHASS nucleotide sequences of the present invention, and fragments and variants thereof, can be used as probes and/or primers to identify and/or clone AHASS homologues in other plants. Such probes can be used to detect transcripts or genomic sequences encoding the same or identical polypeptides.

In this manner, methods such as PCR, hybridization, and the like can be used to identify such sequences having substantial identity to the sequences of the present invention. See, for example, Sambrook et al., 1989, Molecular Cloning: Laboratory Manual, 2d ed., Cold Spring Harbor Laboratory Press, Plainview, N.Y., and Innis, et al., 1990, PCR Protocols: A Guide to Methods and Applications, Academic Press, NY. AHASS nucleotide sequences isolated based on their sequence identity to the AHASS nucleotide sequences set forth herein, or to fragments and variants thereof, are encompassed by the present invention.

In a hybridization method, all or part of a known AHASS nucleotide sequence can be used to screen cDNA or genomic libraries. Methods for construction of such cDNA and genomic libraries are generally known in the art and are disclosed in Sambrook et al., 1989, Molecular Cloning: A Laboratory Manual, 2d ed., Cold Spring Harbor Laboratory Press, Plainview, N.Y. The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known AHASS nucleotide sequence disclosed herein. Degenerate primers designed on the basis of conserved nucleotides or amino acid residues in a known AHASS nucleotide sequence or encoded amino acid sequence can additionally be used. The probe typically comprises a region of nucleotide sequence that hybridizes under stringent conditions to at least about 12, preferably about 25, more preferably about 50, 75, 100, 125, 150, 175, 200, 250, 300, 350, 400, 500, 600, 700, 800, 900, 1000, 1200, 1400, 1600, or 1800 consecutive nucleotides of an AHASS nucleotide sequence of the present invention or a fragment or variant thereof. Methods for the preparation of probes for hybridization are generally known in the art and are disclosed in Sambrook et al., 1989, Molecular Cloning: A Laboratory Manual, 2d ed., Cold Spring Harbor Laboratory Press, Plainview, N.Y., which is herein incorporated by reference.

For example, the entire AHASS sequence disclosed herein, or one or more portions thereof, may be used as a probe capable of specifically hybridizing to corresponding AHASS sequences and messenger RNAs. Hybridization techniques include hybridization screening of plated DNA libraries (either plaques or colonies; see, for example, Sambrook et al., 1989, Molecular Cloning: A Laboratory Manual, 2d ed., Cold Spring Harbor Laboratory Press, Plainview, N.Y.).

Hybridization of such sequences may be carried out under stringent conditions. The term “stringent conditions” or “stringent hybridization conditions” refers to conditions under which a probe will hybridize to its target sequence to a detectably greater degree than to other sequences (e.g., at least 2-fold over background). Stringent conditions are sequence-dependent and will be different in different circumstances.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen, 1993, Laboratory Techniques in Biochemistry and Molecular Biology—Hybridization with Nucleic Acid Probes, Part I, Chapter 2, Elsevier, NY; and Ausubel et al., eds., 1995, Current Protocols in Molecular Biology, Chapter 2, Greene Publishing and Wiley-Interscience, NY. Also See Sambrook et al., 1989, Molecular Cloning: A Laboratory Manual, 2d ed., Cold Spring Harbor Laboratory Press, Plainview, N.Y.

It is recognized that the polynucleotide molecules and polypeptides of the present invention encompass polynucleotide molecules and polypeptides comprising a nucleotide or an amino acid sequence that is sufficiently identical to a nucleotide sequence of SEQ ID NO:1 or SEQ ID NO:3 or to an amino acid sequence of SEQ ID NO:2, 4, or 5. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 77%, 80%, 81%, 85%, 95%, or 98% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted. For the present invention, sequence identity/similarity values are preferably from the alignment without gaps of a full-length nucleotide or full-length amino acid sequence of the present invention to a second nucleotide or amino acid sequence.

The determination of percent identity between two sequences can be accomplished using a mathematical algorithm. A preferred, nonlimiting example of a mathematical algorithm utilized for the comparison of two sequences is the algorithm of Karlin and Altschul, 1990, Proc. Natl. Acad. Sci. USA 87:2264, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403. BLAST nucleotide searches can be performed with the NBLAST program, score=100, wordlength=12, to obtain nucleotide sequences homologous to the polynucleotide molecules of the present invention. BLAST protein searches can be performed with the XBLAST program, score=50, wordlength=3, to obtain amino acid sequences homologous to protein molecules of the present invention. To obtain gapped alignments for comparison purposes, Gapped BLAST can be utilized as described in Altschul et al., 1997, Nucleic Acids Res. 25:3389. Alternatively, PSI-Blast can be used to perform an iterated search that detects distant relationships between molecules. See Altschul et al., 1997, supra. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated herein, pairwise percent sequence identities are generated from the alignment of two nucleotide or two amino acid sequences with ClustalX version 1.81 and MEGA (Molecular Evolutionary Genetics Analysis) version 2.1 using the simple p distance model. The term “equivalent program” refers to any sequence comparison program that, for any two sequences in question, generates an alignment having identical nucleotide or amino acid residue matches and an identical percent sequence identity when compared to the corresponding alignment generated by ClustalX version 1.81 and percent identity calculated by MEGA (Molecular Evolutionary Genetics Analysis) version 2.1 using the simple p distance model.

The AHASS nucleotide sequences of the present invention include both the naturally occurring sequences as well as mutant forms. Likewise, the polypeptides of the present invention encompass both naturally occurring polypeptides as well as variations and modified forms thereof. Such variants will continue to possess the desired AHASS activity. Obviously, the mutations that will be made in the DNA encoding the variant must not place the sequence out of reading frame and preferably will not create complementary regions that could produce secondary mRNA structure (See, e.g., EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by AHAS activity assays. See, for example, Singh et al., 1988, Anal. Biochem. 171:173-179, herein incorporated by reference.

Variant nucleotide sequences and proteins also encompass sequences and proteins derived from a mutagenic and recombinogenic procedure such as DNA shuffling. With such a procedure, one or more different AHASS coding sequences can be manipulated to produce a new AHASS protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the AHASS gene of the present invention and other known AHASS genes to obtain a new gene coding for a protein with an improved property of interest, such as an increased Kₘ in the case of an enzyme. Strategies for such DNA shuffling are known in the art. See, for example, Stemmer, 1994, Proc. Natl. Acad. Sci. USA 91:10747-10751; Stemmer, 1994, Nature 370:389-391; Crameri et al., 1997, Nature Biotech. 15:436-438; Moore et al., 1997, J. Mol. Biol. 272:336-347; Zhang et al., 1997, Proc. Natl. Acad. Sci. USA 94:4504-4509; Crameri et al., 1998, Nature 391:288-291; and U.S. Pat. Nos. 5,605,793 and 5,837,458.

The nucleotide sequences of the present invention can be used to isolate corresponding sequences from other organisms, particularly other plants, more particularly other monocots. In this manner, methods such as PCR, hybridization, and the like can be used to identify such sequences based on their sequence homology to the sequences set forth herein. Sequences isolated based on their sequence identity to the entire AHASS sequences set forth herein or to fragments thereof are encompassed by the present invention. Thus, isolated sequences that encode for an AHASS protein and which hybridize under stringent conditions to the sequence disclosed herein, or to fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al., 1989, Molecular Cloning: A Laboratory Manual, 2d ed., Cold Spring Harbor Laboratory Press, Plainview, N.Y. See also Innis et al., eds., 1990, PCR Protocols: A Guide to Methods and Applications, Academic Press, NY; Innis and Gelfand, eds., 1995, PCR Strategies, Academic Press, NY; and Innis and Gelfand, eds., 1999, PCR Methods Manual, Academic Press, NY. Known methods of PCR include, but are not limited to, methods using paired primers, nested primers, single specific primers, degenerate primers, gene-specific primers, vector-specific primers, partially-mismatched primers, and the like.

The AHASS sequences of the present invention also are provided in expression cassettes for expression in a plant of interest. The cassette will include 5′ and 3′ regulatory sequences operably linked to an AHASS nucleotide sequence of the present invention. The term “operably linked” as used here refers to a functional linkage between a promoter and a second sequence, wherein the promoter sequence initiates and mediates transcription of the DNA sequence corresponding to the second sequence. Generally, operably linked means that the nucleic acid or amino acid sequences are linked such that both sequences fulfill the function or activity attributed to the sequence used. In one embodiment, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the AHASS sequence to be under the transcriptional regulation of the regulatory regions. The expression cassette may additionally contain selectable marker genes.

The expression cassette may include in the 5′-3′ direction of transcription, a transcriptional and translational initiation region (i.e., a promoter), an AHASS sequence of the present invention, and a transcriptional and translational termination region (i.e., termination region) functional in plants. The promoter may be native or analogous, or foreign or heterologous, to the plant host and/or to the AHASS sequence of the present invention. Additionally, the promoter may be the natural sequence or alternatively a synthetic sequence. Where the promoter is “foreign” or “heterologous” to the plant host, it refers to the promoter that is not found in the native plant into which the promoter is introduced. Where the promoter is “foreign” or “heterologous” to the AHASS sequence of the present invention, it refers to the promoter that is not the native or naturally occurring promoter for the operably linked AHASS sequence of the present invention. As used herein, a chimeric gene comprises a coding sequence operably linked to a transcription initiation region that is heterologous to the coding sequence.

While it may be preferable to express the sequences using heterologous promoters, the native AHASS or AHASL promoter sequences also may be used. Such constructs would change expression levels of AHASS protein in the plant or plant cell. Thus, the phenotype of the plant or plant cell is altered.

The termination region may be native with the transcriptional initiation region, may be native with the operably linked AHASS sequence of interest, may be native with the plant host, or may be derived from another source (i.e., foreign or heterologous to the promoter, the AHASS sequence of interest, the plant host, or any combination thereof). Convenient termination regions are available from the Ti-plasmid of A. tumefaciens, such as the octopine synthase and nopaline synthase termination regions (See also Guerineau et al., 1991, Mol. Gen. Genet. 262:141-144; Proudfoot, 1991, Cell 64:671-674; Sanfacon et al., 1991, Genes Dev. 5:141-149; Mogen et al., 1990, Plant Cell 2:1261-1272; Munroe et al., 1990, Gene 91:151-158; Ballas et al., 1989, Nucleic Acids Res. 17:7891-7903; and Joshi et al., 1987, Nucleic Acid Res. 15:9627-9639).

Where appropriate, the gene(s) may be optimized for increased expression in the transformed plant. That is, the genes can be synthesized using plant-preferred codons for improved expression (See, for example, Campbell and Gowri, 1990, Plant Physiol. 92:1-11 for a discussion of host-preferred codon usage). Methods are available in the art for synthesizing plant-preferred genes (See, for example, U.S. Pat. Nos. 5,380,831, and 5,436,391, and Murray et al., 1989, Nucleic Acids Res. 17:477-498, herein incorporated by reference).

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

Nucleotide sequences for enhancing gene expression can also be used in the plant expression vectors. These include the introns of the maize AdhI, intron1 gene (Callis et al., 1987, Genes and Development 1:1183-1200), and leader sequences (W-sequence) from the Tobacco Mosaic virus (TMV), Maize Chlorotic Mottle Virus, and Alfalfa Mosaic Virus (Gallie et al., 1987, Nucleic Acid Res. 15:8693-8711 and Skuzeski et al., 1990, Plant Molec. Biol. 15:65-79). The first intron from the shrunkent-1 locus of maize, has been shown to increase expression of genes in chimeric gene constructs. U.S. Pat. Nos. 5,424,412 and 5,593,874 disclose the use of specific introns in gene expression constructs, and Gallie et al., 1994, Plant Physiol. 106:929-939 also have shown that introns are useful for regulating gene expression on a tissue specific basis. To further enhance or to optimize AHASS small subunit gene expression, the plant expression vectors of the present invention may also contain DNA sequences containing matrix attachment regions (MARs). Plant cells transformed with such modified expression systems, then, may exhibit overexpression or constitutive expression of a nucleotide sequence of the present invention.

The expression cassettes may additionally contain 5′ leader sequences in the expression cassette construct. Such leader sequences can act to enhance translation. Translation leaders are known in the art and include: picomavirus leaders, for example, EMCV leader (Encephalomyocarditis 5′ noncoding region) (Elroy-Stein et al., 1989, Proc. Natl. Acad. Sci. USA 86:6126-6130); potyvirus leaders, for example, TEV leader (Tobacco Etch Virus) (Gallie et al., 1995, Gene 165(2):233-238), MDMV leader (Maize Dwarf Mosaic Virus) (Virology 154:9-20), and human immunoglobulin heavy-chain binding protein (BiP) (Macejak et al., 1991, Nature 353:90-94); untranslated leader from the coat protein mRNA of alfalfa mosaic virus (AMV RNA 4) (Jobling et al., 1987, Nature 325:622-625); tobacco mosaic virus leader (TMV) (Gallie et al., 1989, in Molecular Biology of RNA, ed. Cech, Liss, New York, pp. 237-256); and maize chlorotic mottle virus leader (MCMV) (Lommel et al., 1991, Virology 81:382-385). See also, Della-Cioppa et al., 1987, Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, and substitutions, e.g., transitions and transversions, may be involved.

A number of promoters can be used in the practice of the present invention. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al., 1985, Nature 313:810-812); rice actin (McElroy et al., 1990, Plant Cell 2:163-171); ubiquitin (Christensen et al., 1989, Plant Mol. Biol. 12:619-632 and Christensen et al., 1992, Plant Mol. Biol. 18:675-689); pEMU (Last et al., 1991, Theor. Appl. Genet. 81:581-588); MAS (Velten et al., 1984, EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced AHASS expression within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al., 1997, Plant J. 12(2):255-265; Kawamata et al., 1997, Plant Cell Physiol. 38(7):792-803; Hansen et al., 1997, Mol. Gen Genet. 254(3):337-343; Russell et al., 1997, Transgenic Res. 6(2):157-168; Rinehart et al., 1996, Plant Physiol. 112(3):1331-1341; Van Camp et al., 1996, Plant Physiol. 112(2):525-535; Canevascini et al., 1996, Plant Physiol. 112(2):513-524; Yamamoto et al., 1994, Plant Cell Physiol. 35(5):773-778; Lam, 1994, Results Probl. Cell Differ. 20:181-196; Orozco et al., 1993, Plant Mol Biol. 23(6):1129-1138; Matsuoka et al., 1993, Proc Natl. Acad. Sci. USA 90(20):9586-9590; and Guevara-Garcia et al., 1993, Plant J. 4(3):495-505. Such promoters can be modified, if necessary, for weak expression.

In one embodiment, the nucleic acids of interest are targeted to the chloroplast for expression. In this manner, where the nucleic acid of interest is not directly inserted into the chloroplast, the expression cassette will additionally contain a chloroplast-targeting sequence comprising a nucleotide sequence that encodes a chloroplast transit peptide to direct the gene product of interest to the chloroplasts. Such transit peptides are known in the art. See, for example, Von Heijne et al., 1991, Plant Mol. Biol. Rep. 9:104-126; Clark et al., 1989, J. Biol. Chem. 264:17544-17550; Della-Cioppa et al., 1987, Plant Physiol. 84:965-968; Romer et al., 1993, Biochem. Biophys. Res. Commun. 196:1414-1421; and Shah et al., 1986, Science 233:478-481. While the AHASS polypeptides of the present invention include a native chloroplast transit peptide, any chloroplast transit peptide known in art can be fused to the amino acid sequence of a mature AHASS polypeptide of the present invention by operably linking a chloroplast-targeting sequence to the 5′-end of a nucleotide sequence encoding a mature AHASS polypeptide of the present invention.

Chloroplast targeting sequences are known in the art and include the chloroplast small subunit of ribulose-1,5-bisphosphate carboxylase (Rubisco) (de Castro Silva Filho et al., 1996, Plant Mol. Biol. 30:769-780; Schnell et al., 1991, J. Biol. Chem. 266(5):3335-3342); 5-(enolpyruvyl)shikimate-3-phosphate synthase (EPSPS) (Archer et al., 1990, J. Bioenerg. Biomemb. 22(6):789-810); tryptophan synthase (Zhao et al., 1995, J. Biol. Chem. 270(11):6081-6087); plastocyanin (Lawrence et al., 1997, J. Biol. Chem. 272(33):20357-20363); chorismate synthase (Schmidt et al., 1993, J. Biol. Chem. 268(36):27447-27457); and the light harvesting chlorophyll a/b binding protein (LHBP) (Lamppa et al., 1988, J. Biol. Chem. 263:14996-14999). See also Von Heijne et al., 1991, Plant Mol. Biol. Rep. 9:104-126; Clark et al., 1989, J. Biol. Chem. 264:17544-17550; Della-Cioppa et al., 1987, Plant Physiol. 84:965-968; Romer et al., 1993, Biochem. Biophys. Res. Commun. 196:1414-1421; and Shah et al., 1986, Science 233:478-481.

Methods for transformation of chloroplasts are known in the art (See, for example, Svab et al., 1990, Proc. Natl. Acad. Sci. USA 87:8526-8530; Svab and Maliga, 1993, Proc. Natl. Acad. Sci. USA 90:913-917; Svab and Maliga, 1993, EMBO J. 12:601-606). The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al., 1994, Proc. Natl. Acad. Sci. USA 91:7301-7305.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons (See, for example, U.S. Pat. No. 5,380,831, herein incorporated by reference).

As disclosed herein, the AHASS nucleotide sequences of the present invention may be used to enhance the herbicide tolerance of plants that comprise a gene encoding an herbicide-tolerant AHASL polypeptide. Such an AHASL gene may be incorporated in the plant's genome and may be an endogenous gene or a transgene. Additionally, in certain embodiments, the nucleic acid sequences of the present invention can be stacked with any combination of nucleotide sequences of interest in order to produce plants with a desired phenotype. For example, the polynucleotides of the present invention may be stacked with any other polynucleotides encoding polypeptides having pesticidal and/or insecticidal activity, such as, for example, the Bacillus thuringiensis toxic proteins (described in U.S. Pat. Nos. 5,366,892; 5,747,450; 5,737,514; 5,723,756; 5,593,881; and Geiser et al., 1986, Gene 48:109). The combinations generated also can include multiple copies of any one of the polynucleotides of interest.

It is recognized that with these nucleotide sequences, antisense constructions, complementary to at least a portion of the messenger RNA (mRNA) for the AHASS sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention also may be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity (See U.S. Pat. Nos. 5,283,184 and 5,034,323; herein incorporated by reference).

Generally, the expression cassette will comprise a selectable marker gene for the selection of transformed cells. Selectable marker genes are utilized for the selection of transformed cells or tissues. Marker genes include genes encoding polypeptides that confer antibiotic resistance, such as those genes encoding neomycin phosphotransferase II (NEO) and hygromycin phosphotransferase (HPT), as well as genes that encode polypeptides that confer resistance to herbicidal compounds, such as glufosinate ammonium, bromoxynil, imidazolinones, and 2,4-dichlorophenoxyacetate (2,4-D). See generally, Yarranton, 1992, Curr. Opin. Biotech. 3:506-511; Christopherson et al., 1992, Proc. Natl. Acad. Sci. USA 89:6314-6318; Yao et al., 1992, Cell 71:63-72; Reznikoff, 1992, Mol. Microbiol. 6:2419-2422; Barkley et al., 1980, in The Operon, pp. 177-220; Hu et al., 1987, Cell 48:555-566; Brown et al., 1987, Cell 49:603-612; Figge et al., 1988, Cell 52:713-722; Deuschle et al., 1989, Proc. Natl. Acad. Aci. USA 86:5400-5404; Fuerst et al., 1989, Proc. Natl. Acad. Sci. USA 86:2549-2553; Deuschle et al., 1990, Science 248:480-483; Gossen, 1993, Ph.D. Thesis, University of Heidelberg, Reines et al., 1993, Proc. Natl. Acad. Sci. USA 90:1917-1921; Labow et al., 1990, Mol. Cell. Biol. 10:3343-3356; Zambretti et al., 1992, Proc. Natl. Acad. Sci. USA 89:3952-3956; Baim et al., 1991, Proc. Natl. Acad. Sci. USA 88:5072-5076; Wyborsid et al., 1991, Nucleic Acids Res. 19:4647-4653; Hillenand-Wissman, 1989, Topics Mol. Struc. Biol. 10:143-162; Degenkolb et al, 1991, Antimicrob. Agents Chemother. 35:1591-1595; Kleinschnidt et al., 1988, Biochemistry 27:1094-1104; Bonin, 1993, Ph.D. Thesis, University of Heidelberg; Gossen et al., 1992, Proc. Natl. Acad. Sci. USA 89:5547-5551; Oliva et al., 1992, Antimicrob. Agents Chemother. 36:913-919; Hlavka et al., 1985, Handbook of Experimental Pharmacology, Vol. 78, Springer-Verlag, Berlin; Gill et al., 1988, Nature 334:721-724. Such disclosures are herein incorporated by reference.

The above list of selectable marker genes is not meant to be limiting. Any selectable marker gene can be used in the present invention.

The isolated polynucleotide molecules encoding the AHASS polypeptides can be used in vectors to transform plants so the plants produced have enhanced tolerance to herbicides, particularly imidazolinone herbicides. The isolated polynucleotide molecules encoding the AHASS polypeptides can be used in vectors alone or in combination with a nucleotide sequence encoding the large subunit of the AHAS enzyme in conferring herbicide resistance in plants (See, U.S. Pat. No. 6,348,643; which is hereby incorporated herein in its entirety by reference).

An AHASS nucleotide sequence of the present invention also can be used in combination with an AHASL nucleotide sequence as a marker for selecting transformed plant cells, plant tissues, and plants. Any gene of interest can be incorporated in vectors comprising nucleotide sequences encoding the AHASS and AHASL polypeptides. The vectors can be introduced into plant cells or tissues that are susceptible to AHAS-inhibiting herbicides. Transformed plants, plant tissues, and plant cells containing these vectors may be selected in the presence of herbicides using standard techniques known in the art.

The present invention also provides a plant expression vector comprising a promoter that drives expression in a plant operably linked to an isolated AHASS polynucleotide molecule of the present invention. The isolated polynucleotide molecule comprises a nucleotide sequence encoding a monocot AHASS polypeptide, particularly an AHASS polypeptide comprising an amino sequence that is set forth in SEQ ID NO:2, SEQ ID NO:4, or SEQ ID NO:5, or a functional fragment or variant thereof. The plant expression vector of the present invention does not depend on a particular promoter, only that such a promoter is capable of driving gene expression in a plant cell. Preferred promoters include but are not limited to constitutive promoters and tissue-preferred promoters.

In another embodiment, the plant expression vector comprises: a promoter of a eukaryotic AHASL gene operably linked to a nucleotide sequence encoding the AHASL polypeptide, and a promoter that is capable of driving expression in a plant cell operably linked to an AHASS nucleotide sequence of the present invention, wherein the AHASS nucleotide sequence is selected from group consisting of the nucleotide sequences set forth in SEQ ID NO:1 and SEQ ID NO:3, nucleotide sequences encoding the amino acid sequences set forth in SEQ ID NO:2, SEQ ID NO:4, and SEQ ID NO:5, and fragments and variants thereof that encode a mature AHASS polypeptide comprising AHASS activity.

Such mature AHASS polypeptides are capable of increasing the AHAS activity of at least one AHASL polypeptide when such AHASS and AHASL polypeptides are in the presence of each other, as compared to the AHAS activity of the AHASL polypeptide in the absence of the AHASS polypeptide.

In yet another embodiment, the plant expression vector for expressing a heterologous AHAS gene in a plant comprises a plant promoter operably linked to a nucleotide sequence that encodes a fusion polypeptide comprising the amino acid sequence of mature AHASL polypeptide fused to the amino acid sequence of a AHASS polypeptide. Such a polynucleotide construct comprises a nucleotide sequence that encodes a mature AHASL polypeptide operably linked to an AHASS nucleotide sequence of the present invention.

As used herein, the term “operably linked” in the context of such a polynucleotide encoding a fusion polypeptide refers to a first nucleotide sequence encoding a first amino acid sequence that is ligated or fused to a second nucleotide sequence encoding a second amino acid sequence in such a manner that the fused amino acid sequence that is encoded by the fused nucleotide sequence comprises the first and second amino acid sequences. It is recognized that a polynucleotide construct encoding a fusion polypeptide of the present invention can also comprise additional nucleotide sequences and that such additional nucleotide sequences can be located 5′ of the first coding sequence, 3′ of the second coding sequence, or between the first and second coding sequences. It is further recognized that in certain embodiments of the present invention, it may be desirable to include in such a fused nucleotide sequence encoding a fusion polypeptide an additional nucleotide sequence that encodes a linker amino acid sequence. In the resulting fusion polypeptide, the linker amino acid sequence will be located between the first and second amino acid sequences. It is recognized that it can be desirable to have such a linker amino acid sequence to allow for optimal interaction between the portions of the fusion polypeptide corresponding to the first and second amino acid sequences. Such a linker amino acid sequence can comprise 1, 2, 3, 4, 5, 6, 7, 8, 9, 10, 15, 20, 25, 30, 40, 50, 75, 100, or more amino acids.

When “operably linked” is used in reference to the combination of two amino acid sequences to form a fusion protein, it is intended that the two amino acid sequences are fused or joined so as to form a single continuous amino acid sequence such that both sequences fulfill the function or activity attributed to the sequence used. In one embodiment, such a fusion protein is the translation product of a single continuous nucleotide sequence that comprises a first nucleotide sequence operably linked to a second nucleotide sequence. The first nucleotide sequence encodes the first amino acid sequence, and the second nucleotide sequence encodes the second amino acid sequence. The fusion polypeptide is then produced as the translation product of the single continuous nucleotide sequence.

In another embodiment, the plant expression vector comprises a promoter that is capable of driving gene expression in a plant cell operably linked to a polynucleotide encoding a fusion polypeptide comprising the amino acid sequence of a mature AHASL polypeptide and the amino acid sequence of a mature AHASS polypeptide of the present invention. Thus, the fusion polypeptide is comprised of two domains, an AHASL domain and a AHASS domain. Such a fusion polypeptide may comprise from the N-terminal end, the AHASL domain followed by the AHASS domain, or alternatively, the AHASS domain followed by the AHASL domain. In addition, the fusion polypeptide can further comprise an amino sequence of a linker region. In such a fusion polypeptide, the linker region is situated between the AHASL and AHASS domains. If desired, for chloroplasts expression, the polynucleotide encoding the fusion polypeptide further comprises a chloroplast-targeting sequence encoding a chloroplast transit peptide. Such a chloroplast transit peptide may be selected from a group consisting of the chloroplast transit peptides from the native AHASS or AHASL polypeptides of the fusion polypeptide or any other chloroplast transit peptides known in the art. It is recognized that such a chloroplast transit peptide is typically at the N-terminal end of a protein.

The AHASS nucleotide sequences of the present invention may be used to produce tethered AHAS enzymes, which comprise the AHASL-AHASS fusion polypeptides of the present invention. For example, in an embodiment of the present invention, a first polynucleotide molecule encoding an AHASS polypeptide of the present invention is translationally coupled to a second polynucleotide molecule encoding the amino acid sequence of a eukaryotic AHASL protein via a linker nucleotide sequence encoding a linker region (or linker polypeptide), such as polyglycine (polyGly). That is, the linker nucleotide sequence is operably linked to the 3′ end of the first nucleotide sequence and the 5′ end of the second nucleotide sequence, so as to encode a polypeptide comprising in series the amino acid sequence of the AHASS polypeptide, the amino acid sequence of the linker region, and the amino acid sequence AHASL polypeptide. An alternative positioning involves switching the mature coding sequences of the large and small subunits about the linker region transcript with the small subunit transit sequence. The present invention does not depend on the linker regions having a particular number of amino acids, only that the fusion polypeptide has AHAS activity, preferably a higher level of AHAS activity than the corresponding AHASL polypeptide in the absence of the corresponding AHASS polypeptide.

It is recognized that tethered AHAS enzymes may be used to enhance herbicide tolerance by keeping the large and small AHAS subunit domains in close proximity to each other. It has been shown with the E. coli AHAS enzyme that the association between large and small subunits is loose. It was estimated in E. coli that at a concentration of 10⁻⁷ M for each subunit, the large subunits are only half associated as the α₂β₂ active holoenzyme (Sella et al., 1993, J. Bacteriology 175:5339-5343).

It is recognized that highest activity is achieved when there is a molar excess of the AHASS protein relative to the molar concentration of the AHASL protein. Since it has been determined that the AHAS enzyme is most stable and active when both subunits are associated (Weinstock et al., 1992, J. Bacteriology, 174:5560-5566, Sella et al., 1993, J. Bacteriology 175:5339-5343), a highly active and stable enzyme may be created by fusing the two subunits into a single polypeptide. Tethered polypeptides have been shown to function properly. Gilbert et al. expressed two tethered oligosacharide synthetic enzymes in E. coli to produce an enzyme that was functional, stable in vitro, and soluble (Gilbert et al., 1998, Nature Biotechnology 16: 769-772).

Expression of both the large and small subunits of AHAS as a single polypeptide from a single nucleotide construct also has advantages for producing transgenic herbicide-tolerant crops. The use of a single gene to transform and breed plants into elite crop lines is easier and more advantageous than when two or more genes are used.

A plant expression vector that contains two polynucleotide constructs—one encoding an AHASL polypeptide and the other encoding an AHASS polypeptide—can be constructed. In this manner, the two genes segregate as a single locus, making breeding of herbicide tolerant crops easier. Alternatively, the large and small subunit can be fused into a single gene expressed from a single promoter. The fusion polypeptide would have elevated levels of AHAS activity and herbicide tolerance. The large subunit of AHAS can be of a wild type sequence (if resistance is conferred in the presence of an independent or fused small subunit), or may be a mutant large subunit that in itself has some level of resistance to herbicides. The presence of the small subunit can enhance the activity of the large subunit, enhance the herbicide tolerance of the large subunit, increase the stability of the enzyme when expressed in vivo, and/or increase resistance to large subunit to degradation. The small subunit would in this manner elevate the tolerance of the plant/crop to an imidazolinone or other herbicide. The elevated tolerance would permit the application and/or increase the safety of weed-controlling rates of herbicide without phytotoxicity to the transformed plant. Ideally, the tolerance conferred would elevate tolerance to herbicides that are known to interfere with AHAS such as, for example, imidazolinone and sulfonylurea herbicides.

The association of large and small subunits appears to be highly specific in prokaryotes. E. coli, for example, has three AHASL isozymes and three AHASS isozymes. Each AHASL isozyme specifically associates with only one of the AHASS isozymes, even though all subunits are expressed in the same organism (Weinstock et al., 1992, J. Bacteriology, 174:5560-5566). However, little is known about the specificity of interactions between eukaryotic AHASL and AHASS proteins from the same or different species or from different isozyme pairs of the same species.

The AHASS polypeptides of the present invention can be purified from, for example, maize, rice, and wheat plants and can be used in compositions. Also, an isolated polynucleotide molecule encoding an AHASS protein of the present invention can be used to express an AHASS polypeptide of the present invention in a microbe such as E. coli. The expressed AHASS polypeptide can be purified from extracts of E. coli by any method known to those of ordinary skill in the art.

The present invention also relates to a method for producing a transgenic plant, which is resistant to an herbicide. Such a method comprises transforming a plant with a plant expression vector comprising a promoter that drives expression in a plant operably linked to an isolated polynucleotide molecule of the present invention. The isolated polynucleotide molecule comprises a nucleotide sequence encoding a monocot AHASS polypeptide, particularly an AHASS polypeptide comprising an amino acid sequence selected from the group consisting of: an amino sequence that is set forth in SEQ ID NO:2, SEQ ID NO:4, or SEQ ID NO:5; amino acids 77-483 of the amino acid sequence set forth in SEQ ID NO:2, amino acids 64-471 of the amino acid sequence set forth in SEQ ID NO:4, and amino acids 74-481 of the amino acid sequence set forth in SEQ ID NO:5; or a functional fragment or variant thereof.

The present invention also relates to a method for conferring herbicide tolerance to a plant cell. The method comprises co-transforming the plant cell with a first plant expression vector comprising a first plant expressible promoter operably linked to a nucleotide sequence encoding an AHASL polypeptide and a second plant expression vector comprising a second plant expressible promoter operably linked to a nucleotide sequence encoding an AHASS polypeptide of the present invention. Preferably, the nucleotide sequence encoding the AHASL polypeptide encodes a eukaryotic AHASL polypeptide. In one embodiment, the nucleotide sequence encoding the AHASL polypeptide encodes a plant AHASL polypeptide. In another embodiment, the nucleotide sequence encoding the AHASL protein encodes a monocot AHASL polypeptide. In yet another embodiment, the nucleotide sequence encoding the AHASL polypeptide encodes an AHASL polypeptide for which it is known that AHAS activity is enhanced by the AHASS polypeptide of the present invention.

The present invention further relates to a method for enhancing the herbicide tolerance of a transgenic plant that expresses a gene encoding an AHASL polypeptide or a mutant or variant thereof. Such a method comprises transforming the transgenic plant with an AHASS polynucleotide molecule of the present invention. Preferably, the polynucleotide molecule is operably linked to a promoter that is capable of driving gene expression in a plant or in at least one cell thereof.

The present invention also provides methods for enhancing herbicide resistance in the progeny plants of an herbicide-resistant plant. The method comprises somatically or sexually crossing the plant whose genetic complement comprises a nucleotide sequence encoding an herbicide-resistant eukaryotic AHASL polypeptide with a plant transformed with a polynucleotide molecule encoding an AHASS polypeptide of the present invention and selecting for those progeny plants which exhibit enhanced herbicide resistance. In one embodiment, the selected progeny comprise the polynucleotide molecule encoding the AHASS polypeptide of the present invention stably incorporated in their genomes. Such a progeny plant comprises enhanced resistance to at least one herbicide, when compared to the herbicide resistance of a wild type variety of the plant

The present invention also provides transgenic plants and progeny plants produced by the methods of the present invention, which plants exhibit enhanced resistance to an herbicide that interferes with the AHAS enzyme. The compositions and methods of the present invention may be used to enhance the resistance of a plant or host cell to any class of AHAS inhibitors, including, but not limited to, imidazolinones and sulfonylureas: triazaolopyrimides (chloransulam-methyl, florasulam, diclosulam, metosulam, flumetsulam); pyrimidinyl(thio)benzoates (pyriminobac-methyl, pyrithiobac-Na, pyriftalid, pyribezoxim, bispyribac-Na); and sulfonylamino-carbonyl-triazolinones (flucarbenzone-Na, prooxycarbazone-Na). Preferably, the herbicides of the present invention are those that are used in agriculture such as, for example, imidazolinones, sulfonylureas, chloransulam-methyl, and florasulam. In one embodiment of the present invention, the herbicides are commercially available herbicide products comprising an imidazolinone herbicide including, but not limited to, Backdraft™, Beyond™ Herbicide, Cadre®, Extreme®, Lightning® Herbicide, Pursuit®, Raptor®, and Sceptor®.

Some embodiments of the present invention involve the use of nucleotide sequences encoding AHASL polypeptides. Such nucleotide sequences are known in the art. The present invention does not depend on a particular nucleotide sequence encoding a particular AHASL polypeptide, only that the activity of such an AHASL polypeptide is capable of being enhanced or increased by an AHASS polypeptide of the present invention. Preferably, the nucleotide sequence encodes a eukaryotic AHASL polypeptide. More preferably, the nucleotide sequence encodes a plant AHASL polypeptide. Nucleotide sequences encoding AHASL polypeptides include those set forth in Accession Numbers AAR06607.1 (Camelina microcarpa), AAK68759.1 (Arabidopsis thaliana), AAK50821.1 (Amaranthus powellii) CAA87083.1 (Gossypium hirsutum), CAA87084.1 (Gossypium hirsutum), CAA18088.1 (Papaver rhoeas), BAB20812.1 (Oryza sativa), AAG40279.1 (Solanum ptycanthum), AAG53548.1 (Triticum aestivum), AAG53550.1 (Triticum aestivum), AAM03119.1 (Bromus tectorum), and AAC14572.1 (Hordeum vulgare).

The AHASS polynucleotides of the present invention may be used in methods for enhancing the tolerance of herbicide-tolerant plants. In particular, such herbicide-tolerant plants comprise an herbicide-tolerant or herbicide resistant AHASL polypeptide. Such herbicide-tolerant plants include both plants transformed with an herbicide-tolerant AHASL nucleotide sequence and plants that comprise in their genomes an endogenous gene that encodes an herbicide-tolerant AHASL polypeptide. Nucleotide sequences encoding herbicide-tolerant AHASL polypeptides and herbicide-tolerant plants comprising an endogenous gene that encodes an herbicide-tolerant AHASL polypeptide are known in the art See, for example, U.S. Pat. Nos. 5,013,659, 5,731,180, 5,767,361, 5,545,822, 5,736,629, 5,773,703, 5,773,704, 5,952,553, and 6,274,796; all of which are hereby incorporated by reference in their entirety.

Numerous plant transformation vectors and methods for transforming plants are available. See, for example, An et al., 1986, Plant Pysiol., 81:301-305; Fry et al., 1987, Plant Cell Rep. 6:321-325; Block, 1988, Theor. Appl. Genet.76:767-774; Hinchee et al., 1990, Stadler. Genet. Symp. 203-212; Cousins et al., 1991, Aust. J. Plant Physiol. 18:481-494; Chee and Slightom, 1992, Gene. 118:255-260; Christou et al., 1992, Trends. Biotechnol. 10:239-246; D'Halluin et al., 1992, Bio/Technol. 10:309-314; Dhir et al., 1992, Plant Physiol. 99:81-88; Casas et al., 1993, Proc. Nat. Acad Sci. USA 90:11212-11216; Christou, 1993, In Vitro Cell. Dev. Biol.-Plant; 29P:119-124; Davies et al., 1993, Plant Cell Rep. 12:180-183; Dong and Mchughen, 1993, Plant Sci. 91:139-148; Franklin and Trieu, 1993, Plant. Physiol. 102:167; Golovkin et al., 1993, Plant Sci. 90:41-52; Guo Chin Sci. Bull. 38:2072-2078; Asano et al., 1994, Plant Cell Rep. 13; Ayeres and Park, 1994, Crit. Rev. Plant. Sci. 13:219-239; Barcelo et al., 1994, Plant. J. 5:583-592; Becker et al., 1994, Plant. J. 5:299-307; Borkowska et al., 1994, Acta. Physiol Plant. 16:225-230; Christou, 1994, Agro. Food. Ind. Hi Tech. 5: 17-27; Eapen et al. (1994) Plant Cell Rep. 13:582-586; Hartman, et al. (1994) Bio-Technology 12: 919923; Ritala et al., 1994, Plant. Mol. Biol. 24:317-325; and Wan and Lemaux, 1994, Plant Physiol. 104:3748.

Certain methods of the present invention involve introducing a polynucleotide construct into a plant. As used herein, the term “introducing” refers to presenting to the plant the polynucleotide construct in such a manner that the construct gains access to the interior of a cell of the plant. The methods of the present invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

As used herein, the term “stable transformation” refers to a transformation method wherein the polynucleotide construct introduced into a plant integrates into the genome of the plant and is capable of being inherited by progeny thereof. As used herein, the term “transient transformation” refers to a transformation method wherein a polynucleotide construct introduced into a plant does not integrate into the genome of the plant.

For the transformation of plants and plant cells, the nucleotide sequences of the present invention are inserted using standard techniques into any vector known in the art that is suitable for expression of the nucleotide sequences in a plant or plant cell. The selection of the vector depends on the preferred transformation technique and the target plant species to be transformed. In a preferred embodiment, an AHASS nucleotide sequence is operably linked to a plant promoter that is known for high-level expression in a plant cell, and this construct is then introduced into a plant that comprises in its genome an herbicide-resistant AHASL allele. Such an herbicide resistant AHASL allele can be native or endogenous to the plant genome or can be introduced into the plant genome by any plant transformation method known in the art In this manner, the effectiveness of the herbicide resistance gene (AHASL) may be enhanced by stabilization or activation of the large subunit protein. This method can be applied to any plant species; however, it is most beneficial when applied to crop plants, particularly crop plants that are typically grown in the presence of an herbicide.

Methodologies for constructing plant expression cassettes and for introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake (See, e.g., U.S. Pat. No. 5,405,765 to Vasil et al.; Bilang et al., 1991, Gene 100: 247-250; Scheid et al., 1991, Mol. Gen. Genet., 228: 104-112; Guerche et al., 1987, Plant Science 52: 111-116; Neuhause et al., 1987, Theor. Appl. Genet. 75: 30-36; Klein et al., 1987, Nature 327: 70-73; Howell et al., 1980, Science 208:1265; Horsch et al., 1985, Science 227: 1229-1231; DeBlock et al., 1989, Plant Physiology 91: 694-701; Weissbach and Weissbach, eds., 1988, Methods for Plant Molecular Biology, Academic Press, Inc. and Schuler and Zielinski, eds., 1989, Methods in Plant Molecular Biology, Academic Press, Inc.) The method of transformation depends upon the plant cell to be transformed, stability of vectors used, expression level of gene products and other parameters.

Other suitable methods of introducing a nucleotide sequence into a plant cell and subsequently insertion into the plant genome include microinjection as described by Crossway et al. (1986, Biotechniques 4:320-334), electroporation as described by Riggs et al. (1986, Proc. Natl. Acad. Sci. USA 83:5602-5606); Agrobacterium-mediated transformation as described by Townsend et al. (U.S. Pat. No. 5,563,055) and Zhao et al. (U.S. Pat. No. 5,981,840); direct gene transfer as described by Paszkowski et al. (1984, EMBO J. 3:2717-2722); ballistic particle acceleration as described in, for example, Sanford et al. (U.S. Pat. No. 4,945,050), Tomes et al. (U.S. Pat. No. 5,879,918), Tomes et al. (U.S. Pat. No. 5,886,244), Bidney et al. (U.S. Pat. No. 5,932,782), Tomes et al. (1995, “Direct DNA Transfer into Intact Plant Cells via Microprojectile Bombardment,” in Plant Cell, Tissue, and Organ Culture: Fundamental Methods, ed. Gamborg and Phillips, Springer-Verlag, Berlin; McCabe et al., 1988, Biotechnology 6:923-926); and Lecl transformation (WO 00/28058). Also see, Weissinger et al., 1988, Ann. Rev. Genet. 22:421-477; Sanford et al., 1987, Particulate Science and Technology 5:27-37 (onion); Christou et al., 1988, Plant Physiol. 87:671-674 (soybean); McCabe et al., 1988, Bio/Technology 6:923-926 (soybean); Finer and McMullen, 1991, In Vitro Cell Dev. Biol. 27P: 175-182 (soybean); Singh et al., 1998, Theor. Appl. Genet. 96:319-324 (soybean); Datta et al., 1990, Biotechnology 8:736-740 (rice); Klein et al., 1988, Proc. Natl. Acad. Sci. USA 85:4305-4309 (maize); Klein et al., 1988, Biotechnology 6:559-563 (maize); Tomes, U.S. Pat. No. 5,240,855; Buising et al., U.S. Pat. Nos. 5,322,783 and 5,324,646; Tomes et al., 1995, “Direct DNA Transfer into Intact Plant Cells via Microprojectile Bombardment,” in Plant Cell, Tissue, and Organ Culture: Fundamental Methods, ed. Gamborg, Springer-Verlag, Berlin, (maize); Klein et al., 1988, Plant Physiol. 91:440-444 (maize); Fromm et al., 1990, Biotechnology 8:833-839 (maize); Hooykaas-Van Slogteren et al., 1984, Nature (London) 311:763-764; Bowen et al., U.S. Pat. No. 5,736,369 (cereals); Bytebier et al., 1987, Proc. Natl. Acad. Sci. USA 84:5345-5349 (Liliaceae); De Wet et al., 1985, in The Experimental Manipulation of Ovule Tissues, ed. Chapman et al., Longman, N.Y., pp. 197-209 (pollen); Kaeppler et al., 1990, Plant Cell Reports 9:415-418 and Kaeppler et al., 1992, Theor. Appl. Genet. 84:560-566 (whisker-mediated transformation); D'Halluin et al., 1992, Plant Cell 4:1495-1505 (electroporation); Li et al., 1993, Plant Cell Reports 12:250-255 and Christou and Ford, 1995, Annals of Botany 75:407-413 (rice); Osjoda et al., 1996, Nature Biotechnology 14:745-750 (maize via Agrobacterium tumefaciens); all of which are herein incorporated by reference.

The polynucleotides of the present invention also may be introduced into a plant by contacting the plant with a virus or viral nucleic acids. Generally, such methods involve incorporating a polynucleotide construct of the present invention within a viral DNA or RNA molecule. It is recognized that an AHASS polypeptide of the present invention may initially be synthesized as part of a viral polyprotein, which later may be processed by proteolysis in vivo or in vitro to produce the desired recombinant AHASS polypeptide. Further, it is recognized that promoters of the present invention also encompass promoters utilized for transcription by viral RNA polymerases. Methods for introducing polynucleotide constructs into plants and expressing a protein encoded therein, involving viral DNA or RNA molecules, are known in the art (See, for example, U.S. Pat. Nos. 5,889,191, 5,889,190, 5,866,785, 5,589,367 and 5,316,931; herein incorporated by reference).

Cells of the present invention in which the AHASS polynucleotide has been introduced may be grown into plants in accordance with conventional ways (See, for example, McCormick et al., 1986, Plant Cell Reports 5:81-84). These plants may then be grown, and either pollinated with the same transformed strain or different strains, and the resulting hybrid having constitutive expression of the desired phenotypic characteristic may be identified. Two or more generations may be grown to ensure that expression of the desired phenotypic characteristic is stably maintained and inherited, and then seeds may be harvested to ensure expression of the desired phenotypic characteristic has been achieved. In this manner, the present invention provides a transformed seed (also referred to as “transgenic seed”) having an AHASS polynucleotide construct of the present invention. In one embodiment, the AHASS polynucleotide of the present invention is stably incorporated into a plant genome.

The present invention may be used for transformation of any plant species, including, but not limited to, monocots and dicots. Examples of plant species of interest include, but are not limited to, corn or maize (Zea mays), Brassica sp. (e.g., B. napus, B. rapa, B. juncea), particularly those Brassica species useful as sources of seed oil, alfalfa (Medicago saliva), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum, T. Turgidum ssp. durum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. In one embodiment, plants of the present invention are crop plants (for example, corn, rice, wheat, sugar beet, alfalfa, sunflower, Brassica, soybean, cotton, safflower, peanut, sorghum, millet, tobacco, etc.), preferably grain plants (for example, corn, rice, wheat, barley, sorghum, rye, triticale, etc.), more preferably corn, rice, and wheat plants.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

### EXAMPLES

### Example 1

### Identification of the Full-length AHASS Nucleotide Sequences from Maize, Rice, and Wheat

Total RNA was extracted from Maize leaf tissue (cultivar 3394) using Concert™ plant RNA extraction solution (Invitrogen Corp., Carlsbad, Calif., USA). This total RNA pool served as the source RNA for the production of a first strand cDNA library with Invitrogen's Gene Racer (RLM-RACE) kit. Primers used for rapid amplification of cDNA ends (RACE) were designed, targeting the 5′ region of a less than full length public domain cDNA sequence (ZmAHASS1a: Accession No. AY105043). This partial sequence has a sequencing error that destroys the ORF of the cDNA. Sequence comparison of translated AHASS1 cDNAs with the partial ZmAHASS1a cDNA sequence indicated the base that was likely causing the frame shift. However, until the experimentally derived full-length cDNA was obtained, the identity of the frame shifting base was not certain. The primers employed for the 5′ RACE resolution of the ZmAHASS1a are as follows:

(SEQ ID NO:6)

| | |
|---|---|
| TTCACAAGGATGGAGAGAAGTATGCGAGCGA | gjb 17 |

(SEQ ID NO:7)

| | |
|---|---|
| ACATCACCCCCAGCATTGGATGGTTGA | gjb 18 |

(SEQ ID NO:8)

| | |
|---|---|
| AAGCAGCAGAAAATCGCCAGAAACGGG | gjb 42 |

(SEQ ID NO:9)

| | |
|---|---|
| AACGCCTCTATCAGGTCTGGGTAAG | gjb 43. |

The 5′ RACE products were TA cloned using Promega's pGem T-easy cloning kit. Four separate plasmid clones were sequenced, and the nucleotide sequence that was determined resolved the experimental start codon of cDNA ZmAHASS1a. Using the experimentally derived start codon coupled with the public partial sequence that designated the stop codon, primers for amplification of the full-length cDNA were designed. PCR was performed amplifying the ZmAHASS1a cDNA from a 1st strand cDNA library derived from the plant tissue mentioned above. Twenty-three independent clones from a pool of four independent cDNA reactions were sequenced and analyzed, confirming the experimental cDNA sequence of ZmAHAS1a and confirming the identity of the frame shifting sequencing error in the published partial cDNA sequence AY105043.

Expressed sequence tags (ESTs) corresponding to the maize and wheat AHAS nucleotide sequences of SEQ ID NOS:1 and 3, respectively, were identified in a proprietary EST database based on homology to known AHASS nucleotide sequences. A full-length maize cDNA clone was then obtained using the rapid amplification of cDNA ends method (RACE) method, particularly the 5′-RACE method (Frohman et al., 1988, Proc. Natl. Acad. Sci. USA 85:8998-9002). The resulting cDNA was sequenced to yield the nucleotide sequence set forth in SEQ ID NO:1.

The wheat amino acid sequence (SEQ ID NO:5) is derived from the predicted amino acid sequences of the nucleotide sequences of several overlapping degenerate ESTs (nucleotide sequences not shown). Contig c5532171 was assembled from six proprietary wheat ESTs and two GenBank submissions of partial sequences (gi2139744 and gi21319X). Contig Express (Informax, Inc., North Bethesda, Md., USA) was used to repeat the above assembly from the original proprietary EST. The assembly obtained spanned the entire gene but contained numerous polymorphisms. These likely represent variations among the three homologous genes in wheat. Thus, there was not 100% identity in the overlaps. The predicted amino acid sequence (representing a consensus) was then aligned with those from ZmAHASS1a and OsAHASS1, and other public sequences and each “unexpected” amino acid was checked by examining original nucleotide sequences used for the consensus sequence.

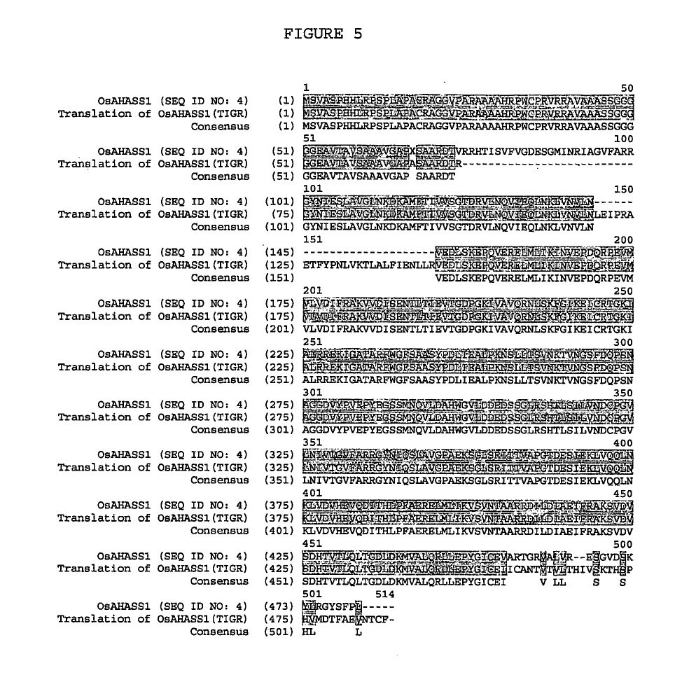

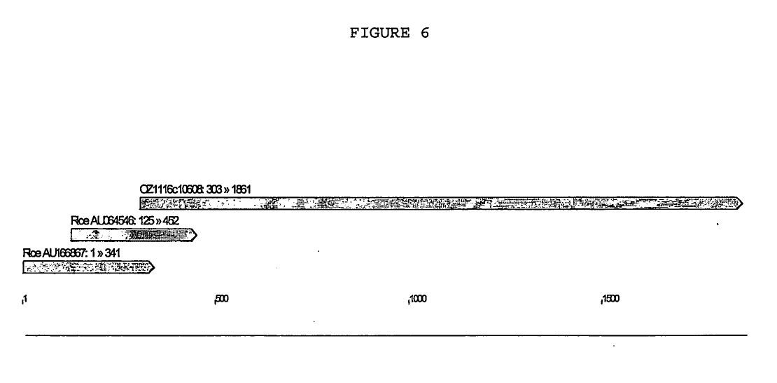

The full-length rice AHASS cDNA was assembled from two public ESTs (Accession numbers: AU064546 and AU166867) and one proprietary contig. The nucleotide sequence of the rice AHASS is set forth in SEQ ID NO:3. The deduced amino acid sequence is set forth in SEQ ID NO:4. The rice AHASS nucleotide and amino acid sequences of the present invention were compared to annotations of the OsAHASS1 genomic DNA that are available from TIGR (The Institute for Genomic Research, 9712 Medical Center Drive, Rockville, Md. 20850; online at www.tigr.org). The TIGR reference numbers for annotations of the OsAHASS1 genomic DNA are TIGR gene temp id: 8351.t03738 and 8351.t03738. However, these annotations were not identical to the full-length rice AHASS nucleotide (SEQ ID NO:3) and amino acid (SEQ ID NO:4) sequences of the present invention. At the nucleotide level, there are two exon differences between the annotation and SEQ ID NO:3. The differences at the amino acid level are depicted in the alignment presented in FIG. 5.

### Example 2

### AHASS Proteins from Maize, Rice, and Wheat

The amino acid sequences of the maize, rice, and wheat AHASS proteins of the present invention are set forth in SEQ ID NOS:2, 4, and 5, respectively. From comparisons with the amino acid sequences of the present invention to other known plant AHASS amino acid sequences, the location of the chloroplast transit peptide was determined for each of the amino acid sequences of the present invention. For the maize AHASS protein, the chloroplast transit peptide corresponds to amino acids 1-76, and the mature protein corresponds to amino acids 77-483 of SEQ ID NO:2. For the rice AHASS protein, the chloroplast transit peptide corresponds to amino acids 1-73, and the mature protein corresponds to amino acids 74-481 of SEQ ID NO:4. For the wheat AHASS protein, the chloroplast transit peptide corresponds to amino acids 1-63, and the mature protein corresponds to amino acids 64-471 of SEQ ID NO:5.

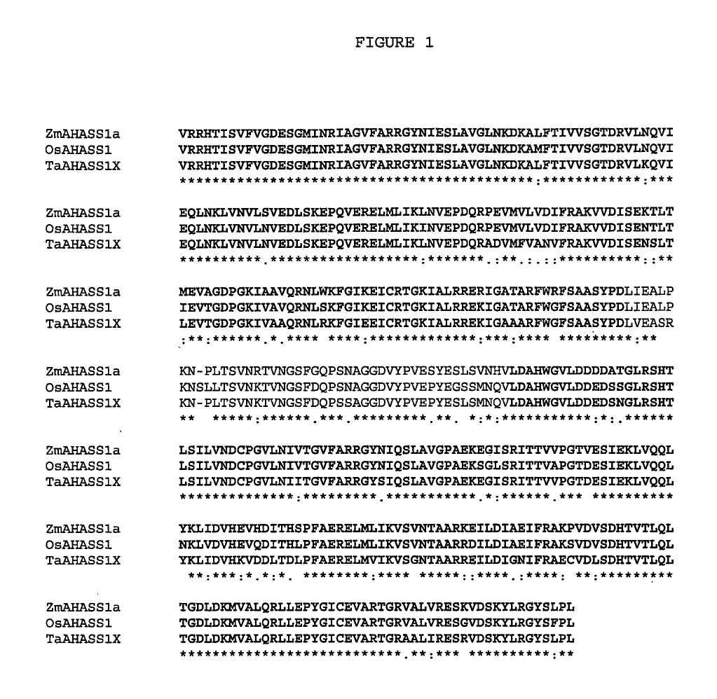

An alignment of the amino acid sequences of the mature AHASS proteins of the present invention is provided in FIG. 1. The three AHAS proteins of the present invention each contain two conserved domains, designated as Domain 1 and Domain 2, which are separated by a variable linker region.

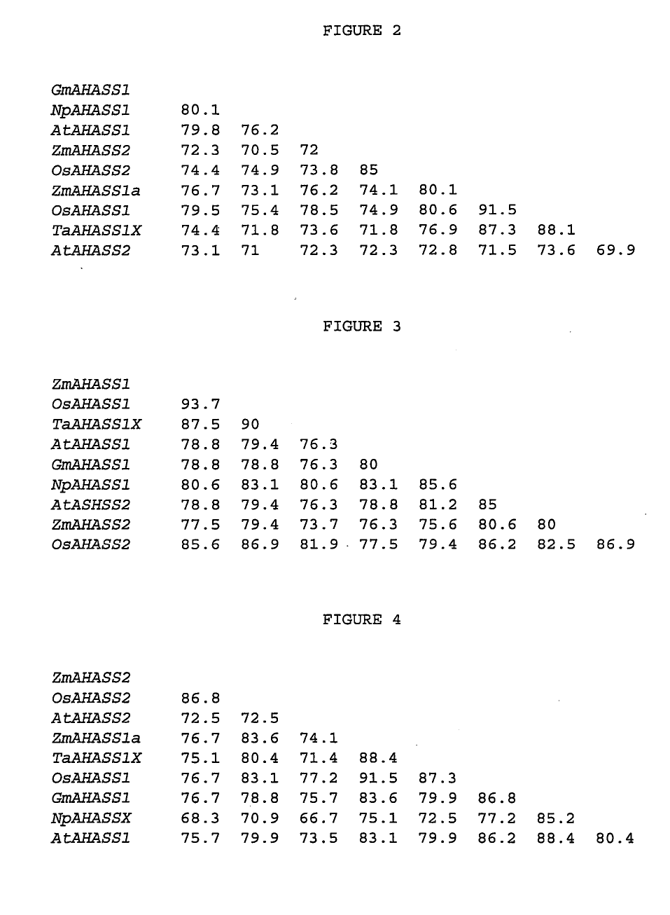

The amino acid sequences of the present invention were compared to other known plant AHASS amino acid sequences. FIG. 2 provides amino acid sequence identities from pairwise comparisons of the mature AHASS proteins of the present invention and known mature AHASS proteins from plants. FIGS. 3 and 4 provide the results of similar comparisons for Domains 1 and 2, respectively.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. Although the foregoing invention has been described in some detail by way of illustration and example for purposes of clarity of understanding, it will be obvious that certain changes and modifications may be practiced within the scope of the appended claims.

We claim:

1. An isolated polynucleotide comprising a nucleotide sequence selected from the group consisting of:
(a) a polynucleotide as defined in SEQ ID NO:1, SEQ ID NO:3; consecutive nucleotides 275-1495 of SEQ ID NO:1, or consecutive nucleotides 342-1565 of SEQ ID NO:3;
(b) a polynucleotide having at least 80% sequence identity with the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has acetohydroxyacid synthase small subunit (AHASS) activity;
(c) a polynucleotide that hybridizes under stringent conditions to the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has AHASS activity;
(d) a polynucleotide encoding a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5;
(e) a polynucleotide encoding a polypeptide having at least 81% sequence identity with the amino acid sequence as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5, wherein the polynucleotide encodes a polypeptide that has AHASS activity;
(f) a polynucleotide encoding a polypeptide having at least 77% sequence identity with the consecutive amino acids 64-471 of SEQ ID NO:5, wherein the polynucleotide encodes a polypeptide that has AHASS activity;
(g) a polynucleotide as defined in SEQ ID NO:10; and
(h) a polynucleotide as defined in SEQ ID NO:11.

2. The isolated polynucleotide of claim 1, wherein the polynucleotide comprises a nucleotide sequence as defined in SEQ ID NO:1, SEQ ID NO:3, consecutive nucleotides 275-1495 of SEQ ID NO:1, or consecutive nucleotides 342-1565 of SEQ ID NO:3.

3. The isolated polynucleotide of claim 1, wherein the polynucleotide comprises a nucleotide sequence having at least 90% sequence identity with the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has AHASS activity.

4. The isolated polynucleotide of claim 1, wherein the polynucleotide comprises a nucleotide sequence encoding a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5.

5. The isolated polynucleotide of claim 1, wherein the polynucleotide comprises a polynucleotide encoding a polypeptide having at least 90% sequence identity with the amino acid sequence as defined in SEQ ID NO:2, SEQ ID NO:4, SEQ ID NO:5, consecutive amino acids 77-483 of SEQ ID NO:2, consecutive amino acids 74-481 of SEQ ID NO:4; or consecutive amino acids 64-471 of SEQ ID NO:5, wherein the polynucleotide encodes a polypeptide that has AHASS activity.

6. The isolated polynucleotide of claim 1, wherein the polynucleotide comprises a polynucleotide as defined in SEQ ID NO:10 or SEQ ID NO:11.

7. The isolated polynucleotide of any one of (a) to (f) of claim 1, wherein the polynucleotide is in an expression cassette comprising a promoter operably linked to the polynucleotide.

8. The isolated polynucleotide of claim 7, wherein the polynucleotide is in a plant expression vector.

9. The isolated polynucleotide of claim 7, wherein the expression cassette further comprises a nucleotide sequence encoding a chloroplast transit peptide operably linked to the polynucleotide.

10. The isolated polynucleotide of claim 7, wherein the promoter is capable of driving expression of the polynucleotide in a host cell selected from the group consisting of a bacterium, a fungal cell, an animal cell, and a plant cell.

11. The isolated polynucleotide of claim 7, wherein the expression cassette is present in a host cell selected from the group consisting of a bacterium, a fungal cell, an animal cell, and a plant cell.

12. The isolated polynucleotide of claim 7, wherein the expression cassette is in a plant.

13. The isolated polynucleotide of claim 8, wherein the plant expression vector further comprises a second polynucleotide construct comprising a second promoter operably linked to a second nucleotide sequence encoding a eukaryotic AHASL polypeptide, wherein both promoters are capable of driving gene expression in a plant cell.

14. The isolated polynucleotide of claim 13, wherein the polynucleotide is selected from the group consisting of: a nucleotide sequence as defined in SEQ ID NO:1, SEQ ID NO:3, consecutive nucleotides 275-1495 of SEQ ID NO:1, or consecutive nucleotides 342-1565 of SEQ ID NO:3; and a nucleotide sequence encoding a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5

15. The isolated polynucleotide of claim 13, wherein the eukaryotic AHASL polypeptide is a plant AHASL polypeptide.

16. The isolated polynucleotide of claim 13, wherein the eukaryotic AHASL polypeptide is an herbicide-tolerant AHASL polypeptide.

17. The isolated polynucleotide of claim 13, wherein the expression vector is in a plant cell.

18. The plant expression vector of claim 12, wherein the expression vector is in a plant.

19. An isolated polypeptide comprising an amino acid sequence selected from the group consisting of:
(a) a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5;
(b) a polypeptide having at least 81% sequence identity with the amino acid sequence as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ-ID NO:5, wherein the polypeptide has acetohydroxyacid synthase small subunit (AHASS) activity;
(c) a polypeptide having at least 77% sequence identity with the consecutive amino acids 64-471 of SEQ ID NO:5, wherein the polypeptide has AHASS activity;
(d) a polypeptide encoded by a polynucleotide having at least 80% sequence identity with a nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polypeptide has AHASS activity; and
(e) a polypeptide encoded by a polynucleotide that hybridizes under stringent conditions to a nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polypeptide has AHASS activity.

20. The isolated polypeptide of claim 19, wherein the polypeptide is defined in SEQ ID NO:2, SEQ ID NO:4, SEQ ID NO:5, consecutive amino acids 77-483 of SEQ ID NO:2, consecutive amino acids 74-481 of SEQ ID NO:4; or consecutive amino acids 64-471 of SEQ ID NO:5.

21. A transgenic plant cell comprising a polynucleotide construct comprising a nucleotide sequence selected from the group consisting of:
(a) a polynucleotide as defined in SEQ ID NO:1, SEQ ID NO:3, consecutive nucleotides 275-1495 of SEQ ID NO:1, or consecutive nucleotides 342-1565 of SEQ ID NO:3;
(b) a polynucleotide having at least 80% sequence identity with the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has acetohydroxyacid synthase small subunit (AHASS) activity;
(c) a polynucleotide that hybridizes under stringent conditions to the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has AHASS activity;
(d) a polynucleotide encoding a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5;
(e) a polynucleotide encoding a polypeptide having at least 81% sequence identity with the amino acid sequence as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5, wherein the polynucleotide encodes a polypeptide that has AHASS activity; and
(f) a polynucleotide encoding a polypeptide having at least 77% sequence identity with the consecutive amino acids 64-471 of SEQ ID NO:5, wherein the polynucleotide encodes a polypeptide that has AHASS activity.

22. The transgenic plant cell of claim 21, wherein the polynucleotide construct is operably linked to a promoter selected from the group consisting of a constitutive promoter and a tissue-preferred promoter.

23. The transgenic plant cell of claim 21, wherein the polynucleotide construct further comprises a second nucleotide sequence encoding a chloroplast transit peptide operably linked to the first nucleotide sequence.

24. The transgenic plant cell of claim 21, wherein the AHAS activity of the transgenic plant cell is increased as compared to a wild type variety of the plant cell.

25. The transgenic plant cell of claim 21, wherein the tolerance of the transgenic plant cell to at least one herbicide is increased as compared to a wild type variety of the plant cell.

26. The transgenic plant cell of claim 21, wherein the transgenic plant cell is a monocot plant cell selected from the group consisting of maize, wheat, rice, barley, rye, oats, triticale, millet, and sorghum.

27. The transgenic plant cell of claim 21, wherein the transgenic plant cell is from a dicot plant cell selected from the group consisting of soybean, cotton, Brassica spp., tobacco, potato, sugar beet, alfalfa, sunflower, safflower, and peanut.

28. The transgenic plant cell of claim 21, wherein the transgenic plant cell is in a plant.

29. The transgenic plant cell of claim 21, wherein the transgenic plant cell is in a seed.

30. A method for enhancing AHAS activity in a plant, comprising introducing a polynucleotide construct into a plant cell and generating from the plant cell a transgenic plant having increased AHAS activity as compared to a wild type variety of the plant, wherein the polynucleotide construct comprises a nucleotide sequence selected from the group consisting of:
(a) a polynucleotide as defined in SEQ ID NO:1, SEQ ID NO:3; consecutive nucleotides 275-1495 of SEQ ID NO:1, or consecutive nucleotides 342-1565 of SEQ ID NO:3;
(b) a polynucleotide having at least 80% sequence identity with the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has acetohydroxyacid synthase small subunit (AHASS) activity;
(c) a polynucleotide that hybridizes under stringent conditions to the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has AHASS activity;
(d) a polynucleotide encoding a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5;
(e) a polynucleotide encoding a polypeptide having at least 81% sequence identity with the amino acid sequence as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5, wherein the polynucleotide encodes a polypeptide that has AHASS activity; and
(f) a polynucleotide encoding a polypeptide having at least 77% sequence identity with the consecutive amino acids 64-471 of SEQ ID NO:5, wherein the polynucleotide encodes a polypeptide that has AHASS activity.

31. The method of claim 30, wherein the transgenic plant has increased tolerance to an herbicide as compared to a wild type variety of the plant.

32. The method of claim 31, wherein the transgenic plant has increased tolerance to an imidazolinone herbicide as compared to a wild type variety of the plant.

33. The method of claim 30, wherein the plant is an herbicide-tolerant plant.

34. The method of claim 33, wherein the plant is an imidazolinone-tolerant plant.

35. The method of claim 33, wherein the plant comprises an herbicide-tolerant acetohydroxyacid synthase large subunit (AHASL) polypeptide.

36. The method of claim 30, wherein the polynucleotide construct further comprises a promoter operably linked to the nucleotide sequence, and wherein the promoter is selected from the group consisting of a constitutive promoter and a tissue-preferred promoter.

37. The method of claim 30, wherein the polynucleotide construct further comprises a polynucleotide sequence encoding an herbicide-tolerant acetohydroxyacid synthase large subunit (AHASL) polypeptide.

38. A transgenic plant having increased AHAS activity as compared to a wild type variety of the plant produced by a method comprising, introducing a polynucleotide construct into a plant cell and generating from the plant cell the transgenic plant, wherein the polynucleotide construct comprises a nucleotide sequence selected from the group consisting of:
(a) a polynucleotide as defined in SEQ ID NO:1, SEQ ID NO:3; consecutive nucleotides 275-1495 of SEQ ID NO: 1, or consecutive nucleotides 342-1565 of SEQ ID NO:3;
(b) a polynucleotide having at least 80% sequence identity with the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has acetohydroxyacid synthase small subunit (AHASS) activity;
(c) a polynucleotide that hybridizes under stringent conditions to the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has AHASS activity;
(d) a polynucleotide sequence encoding a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5;
(e) a polynucleotide encoding a polypeptide having at least 81% sequence identity with the amino acid sequence as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5, wherein the polynucleotide encodes a polypeptide comprising AHASS activity; and
(f) a polynucleotide encoding a polypeptide having at least 77% sequence identity with the consecutive amino acids 64-471 of SEQ ID NO:5, wherein the polynucleotide encodes a polypeptide comprising AHASS activity.

39. The transgenic plant of claim 38, wherein the polynucleotide construct comprises a nucleotide sequence as defined in SEQ ID NO:1, SEQ ID NO:3, consecutive nucleotides 275-1495 of SEQ ID NO:1, or consecutive nucleotides 342-1565 of SEQ ID NO:3.

40. The transgenic plant of claim 38, wherein the polynucleotide construct comprises a nucleotide sequence encoding a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4; or consecutive amino acids 64-471 of SEQ ID NO:5.

41. A method for controlling weeds in the vicinity of a plant, comprising applying an imidazolinone herbicide to the weeds and to the plant, wherein the plant has increased tolerance to the imidazolinone herbicide as compared to a wild type variety of the plant and wherein the plant comprises a polynucleotide construct that comprises a nucleotide sequence selected from the group consisting of:
(a) a polynucleotide as defined in SEQ ID NO:1, SEQ ID NO:3; consecutive nucleotides 275-1495 of SEQ ID NO:1, or consecutive nucleotides 342-1565 of SEQ ID NO:3;
(b) a polynucleotide having at least 80% sequence identity with the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has acetohydroxyacid synthase small subunit (AHASS) activity;
(c) a polynucleotide that hybridizes under stringent conditions to the nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polynucleotide encodes a polypeptide that has AHASS activity;
(d) a polynucleotide sequence encoding a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5;
(e) a polynucleotide encoding a polypeptide having at least 81% sequence identity with the amino acid sequence as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5, wherein the polynucleotide encodes a polypeptide comprising AHASS activity; and
(f) a polynucleotide encoding a polypeptide having at least 77% sequence identity with the consecutive amino acids 64-471 of SEQ ID NO:5, wherein the polynucleotide encodes a polypeptide comprising AHASS activity.

42. A fusion polypeptide comprising an acetohydroxyacid synthase large subunit (AHASL) domain operably linked to an acetohydroxyacid synthase small subunit (AHASS) domain; wherein the fusion polypeptide comprises AHAS activity, wherein the AHASL domain comprises an amino acid sequence of a mature eukaryotic AHASL polypeptide, and wherein the AHASS domain comprises an amino acid sequence of an AHASS polypeptide selected from the group consisting of:
(a) a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5;
(b) a polypeptide having at least 81% sequence identity with the amino acid sequence as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5, wherein the polypeptide has acetohydroxyacid synthase small subunit (AHASS) activity;
(c) a polypeptide having at least 77% sequence identity with the consecutive amino acids 64-471 of SEQ ID NO:5, wherein the polypeptide has AHASS activity;
(d) a polypeptide encoded by a polynucleotide having at least 80% sequence identity with a nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polypeptide has AHASS activity; and
(e) a polypeptide encoded by a polynucleotide that hybridizes under stringent conditions to a nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polypeptide has AHASS activity.

43. The fusion polypeptide of claim 42, wherein the eukaryotic AHASL polypeptide is a plant AHASL polypeptide.

44. The fusion polypeptide of claim 42, further comprising a linker region operably linked between the AHASL domain and the AHASS domain.

45. The fusion polypeptide of claim 42, wherein the AHASL polypeptide and the AHASS polypeptide are from different species.

46. An isolated polynucleotide, wherein the polynucleotide encodes an acetohydroxyacid synthase large subunit (AHASL)-acetohydroxyacid synthase small subunit (AHASS) fusion polypeptide, wherein the AHASL is a eukaryotic AHASL polypeptide, and wherein the AHASS comprises an amino acid sequence selected from the group consisting of:
(a) a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5;
(b) a polypeptide having at least 81% sequence identity with the amino acid sequence as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5, wherein the polypeptide has acetohydroxyacid synthase small subunit (AHASS) activity;
(c) a polypeptide having at least 77% sequence identity with the consecutive amino acids 64-471 of SEQ ID NO:5, wherein the polypeptide has AHASS activity;
(d) a polypeptide encoded by a polynucleotide having at least 80% sequence identity with a nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polypeptide has AHASS activity; and
(e) a polypeptide encoded by a polynucleotide that hybridizes under stringent conditions to a nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polypeptide has AHASS activity.

47. The isolated polynucleotide of claim 46, wherein the AHASS comprises an amino acid sequence as defined in SEQ ID NO:2, SEQ ID NO:4, SEQ ID NO:5, consecutive amino acids 77-483 of SEQ ID NO:2, consecutive amino acids 74-481 of SEQ ID) NO:4, or consecutive amino acids 64-471 of SEQ ID NO:5.

48. The isolated polynucleotide of claim 46, wherein the polynucleotide further comprises an operably linked third nucleotide sequence encoding a linker region.

49. The isolated polynucleotide of claim 46, wherein the polynucleotide further comprises a chloroplast-targeting sequence operably linked to the polynucleotide.

50. The isolated polynucleotide of claim 46, wherein the eukaryotic AHASL polypeptide is a plant AHASL polypeptide.

51. The isolated polynucleotide of claim 46, wherein the eukaryotic AHASL polypeptide is an herbicide-tolerant AHASL polypeptide.

52. The isolated polynucleotide of claim 46, wherein the polynucleotide is in a plant expression vector.

53. The isolated polynucleotide of claim 46, wherein the polynucleotide is in a plant cell.

54. The isolated polynucleotide of claim 46, wherein the polynucleotide is in a seed.

55. A method for producing a transgenic plant having increased AHAS activity comprising, introducing a polynucleotide construct into a plant cell and generating from the transgenic plant cell a transgenic plant having increased AHAS activity as compared to a wild type variety of the plant, wherein the polynucleotide construct encodes an acetohydroxyacid synthase large subunit (AHASL)-acetohydroxyacid synthase small subunit (AHASS) fusion polypeptide, wherein the AHASL is a eukaryotic AHASL polypeptide, and wherein the AHASS comprises an amino acid sequence selected from the group consisting of:
(a) a polypeptide as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5;
(b) a polypeptide having at least 81% sequence identity with the amino acid sequence as defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5, wherein the polypeptide has acetohydroxyacid synthase small subunit (AHASS) activity;
(c) a polypeptide having at least 77% sequence identity with the consecutive amino acids 64-471 of SEQ ID NO:5, wherein the polypeptide has AHASS activity;
(d) a polypeptide encoded by a polynucleotide having at least 80% sequence identity with a nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polypeptide has AHASS activity; and
(e) a polypeptide encoded by a polynucleotide that hybridizes under stringent conditions to a nucleotide sequence as defined in SEQ ID NO:1, SEQ ID NO:3, consecutive nucleotides 275-1495 of SEQ ID NO:1, or consecutive nucleotides 342-1565 of SEQ ID NO:3, wherein the polypeptide has AHASS activity.

56. The method of claim 55, wherein the AHASS polypeptide is defined in SEQ ID NO:2, SEQ ID NO:4, SEQ ID NO:5, consecutive amino acids 77-483 of SEQ ID NO:2, consecutive amino acids 74-481 of SEQ ID NO:4, or consecutive amino acids 64-471 of SEQ ID NO:5.

57. The method of claim 55, wherein the transgenic plant has increased tolerance to an imidazolinone herbicide as compared to a wild type variety of the plant.
